(19) United States
(12) Patent Application Publication
(10) Pub. No.: US 2008/0299568 A1
(43) Pub. Date: Dec. 4, 2008

(54) MATERIALS AND METHODS FOR DETECTION OF HEPATITIS C VIRUS

(76) Inventors: Scott Johnson, Sun Prairie, WI (US); Andrew Newman, Menomonie, WI (US); Elizabeth Mulligan, Madison, WI (US)

Correspondence Address: FOLEY & LARDNER LLP, 150 EAST GILMAN STREET, P.O. BOX 1497, MADISON, WI, 53701-1497, US

(21) Appl. No.: 12/108,061

(22) Filed: Apr. 23, 2008

Related U.S. Application Data
(60) Provisional application No. 60/914,534, filed on Apr. 27, 2007.

(51) Int. Cl. C12Q 1/68

(52) U.S. Cl. 435/6

(57) ABSTRACT

Disclosed are methods and compositions for detecting hepatitis C virus (HCV) in a sample. Typically, the methods include amplifying nucleic acids and detecting signals, such as signals emitted from fluorophores. The signals may be correlated to the presence and/or quantity of the hepatitis C viral genome in the sample. In some embodiments, primers are provided that specifically hybridize to the 3′ non-coding (NC) region of the HCV genome.

### CROSS-REFERENCE TO RELATED APPLICATIONS

This application claims priority to U.S. Provisional Patent Application No. 60/914,534, filed Apr. 27, 2007, which is incorporated by reference in its entirety.

### FIELD OF THE INVENTION

The present invention relates broadly to the detection of pathogens using nucleic acid amplification techniques. In particular, the methods, compositions, and kits relate to detection and quantitation of hepatitis C virus (HCV).

### BACKGROUND OF THE INVENTION

Hepatitis C is a blood-borne, infectious viral disease that is caused by a hepatotropic virus called hepatitis C virus (HCV) that affects an estimated 150-200 million people worldwide. The infection is often asymptomatic, but can frequently causes liver inflammation. Chronic hepatitis can result in cirrhosis (scarring of the liver) and liver cancer.

HCV is spread by contact with an infected person's blood. The symptoms can be treated, and a proportion of patients can be cleared of the virus by a long course of anti-viral medicines. Although modification of diet and early medical intervention are helpful, people with HCV infection often experience only mild symptoms, and consequently do not immediately seek treatment. The ability to detect and quantify HCV during infection is a critical aspect for disease management. Viral titers vary greatly among untreated individuals and those undergoing therapy. Response to treatment is largely gauged by the reduction of viral load.

### SUMMARY OF THE INVENTION

Disclosed herein are methods and compositions relating to the detection and quantification of HCV in patient samples. The disclosed methods allow for reliably, quickly, easily, and inexpensively detecting and quantifying nucleic acids for HCV. Typically, the methods include amplifying nucleic acids and detecting signals, such as signals emitted from fluorophores. The signals may be correlated to the presence and/or quantity of the hepatitis C viral genome in the sample.

In some aspects, the methods provide for testing the presence and quantity of HCV nucleic acid in a sample by reacting a mixture that comprises the sample to be tested, a first primer that is complementary to HCV nucleic acid, wherein the first primer comprises a first label and a first non-natural base (such as iso-C or iso-G); a second primer that is complementary to HCV nucleic acid, and a reporter comprising a second label conjugated to a second non-natural base (such is iso-C or iso-G) that base-pairs with the first non-natural base; and amplifying the HCV nucleic acid in the sample, if present, to generate amplification products, wherein the reporter is incorporated into the amplification products; and observing a signal from the first label, the second label, or both during amplification thereby detecting and quantifying the HCV nucleic acid in the sample, if present.

The primers of the methods may be used to amplify any suitable target nucleic acid. In some embodiments, at least one primer of the reaction mixture is used to amplify HCV nucleic acid. In some embodiments, the primers specifically hybridize to either the 5′ or 3′ NC region of the HCV genome. Optionally, the primers of the methods may be used to amplify a control nucleic acid (e.g., the reaction mixture may include primers for amplifying a control nucleic acid which is present in the sample or which is added to the sample). Optionally, the primers of the methods may be used to simultaneously amplify additional nucleic acids from one or more pathogens or nucleic acids associated with a disease state.

Typically, two specific primers are added to the sample, but the methods are not so limited. The two or more specific primers may comprise a label that is detectable, such as a fluorophore. Suitable fluorophores include, e.g. fluorescein and hexachlorofluoroscein. Each specific primer can include a non-natural nucleotide base such as isocytosine (iso-C) and isoguanine (iso-G). In some embodiments, a mixture of labeled and unlabeled primers having the same sequence is added to the reaction mixture in order to enhance the sensitivity of the assay. For example, the mixture of labeled:unlabeled primer is flexible and may have a ratio of 1:1, 1:2, 2:1, 1:3, 3:1, 1:4, 4:1, 1:5, 5:1, or any fraction thereof. In some embodiments, the mixture may include equal amounts of labeled and unlabeled primer, e.g., 300 nM labeled primer and 300 nM unlabeled primer.

In some embodiments, the methods can further include adding a non-natural nucleotide base in the form of a deoxy-nucleotide triphosphate. Suitable non-natural nucleotide bases include isocytosine and isoguanosine. Typically, the non-natural nucleotide base is complementary to the non-natural nucleotide used in the specific primers. The non-natural nucleotide base can include a fluorescence quencher such as Dabcyl, for example.

In some embodiments, the reaction mixture further includes an amplification mixture. The reaction mixture may include one or more of nucleotides (e.g., dATP, dCTP, dGTP, dTTP, UTP), salts, buffers, surfactants, enzymes, and the like. The reaction mixture may include nucleotide analogs such as nucleotides with thio-substituted phosphates (e.g., a thio analog of at least one of dATP, dCTP, dGTP, dTTP, UTP, or non-natural nucleotides such as deoxy iso-cytidine triphosphate (diCTP) and deoxy iso-guanosine triphosphate (diGTP)) that includes a sulfur atom instead of an oxygen atom in the alpha, beta, or gamma position of the triphosphate). The reaction mixture may include dideoxy analogs of nucleotides (i.e., a 2′,3′-dideoxy analog of at least one of ATP, CTP, GTP, TTP, UTP, or non-natural nucleotides such as iCTP and iGTP). The reaction mixture may include phosphoramidite analogs of nucleotides (e.g., a 3′ phosphoramidite analog of at least one of dATP, dCTP, dGTP, dTTP, and UTP or non-natural nucleotides such as diCTP and diGTP).

The methods and kits can be applied to a wide variety of detection technologies including “real time” or “continuous” detection technologies or “end-point read.”

In some embodiments, the methods of detection are designed to identify all HCV genotypes. In other embodiments, specific genotypes or subtypes of HCV are specifically detected.

### BRIEF DESCRIPTION OF THE DRAWINGS

FIG. 1 is a schematic of the MultiCode® RTx system. Targets are amplified with a standard reverse primer and a forward primer that contains a single iso-C nucleotide and a fluorescent reporter. Amplification is performed in the presence of dabcyl-iso-GTP. Site specific incorporation places the quencher in close proximity to the reporter that leads to a decrease in fluorescence.

FIG. 2 is a schematic representation of HCV with an exploded view of the secondary structure of the 3′ NC region.

FIG. 3 shows the results of a 10-fold dilution series of HCV RNA. FIG. 3A (upper panel) shows the amplification curves for each sample plotted as relative fluorescence units (RFU) versus PCR cycle number. The lower panel shows the melt curve performed after amplication. FIG. 3B is a plot of threshold cycle (C_{T}) versus concentration of HCV RNA.

FIG. 4A shows quantitative results of the MultiCode®-RTx HCV assay compared to Bayer Versant® HCV RNA bDNA 3.0 assay. FIG. 4B shows the MultiCode®-RTx HCV assay compared to the Roche COBAS Amplicor HCV Monitor™ 2.0.

FIG. 5 shows the linearity and sensitivity of the MultiCode®-RTx assay compared to the Bayer Versant® HCV RNA bDNA 3.0 and Roche COBAS Amplicor HCV Monitor™ 2.0 systems.

FIG. 6 shows the detection on various HCV genotypes using MultiCod®-RTx, Bayer Versant® HCV RNA bDNA 3.0, and Roche COBAS Amplicor HCV Monitor™ 2.0 systems. Results are compared pairwise.

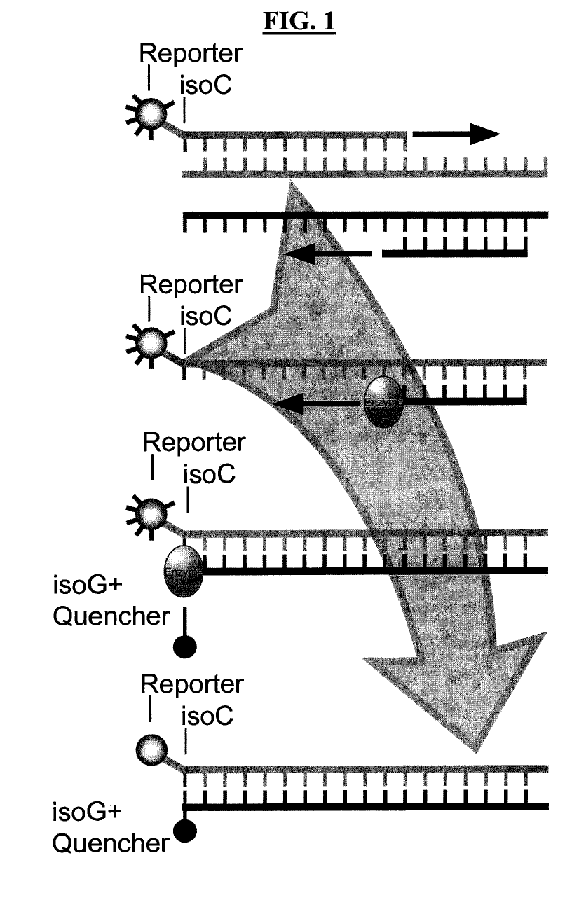

### DETAILED DESCRIPTION OF MODES OF PRACTICING THE INVENTION

Disclosed herein are methods and materials for detecting the presence of hepatitis-C virus (HCV) in a sample and methods of quantifying the viral load in a sample.

Nucleic acid-based methods to quantitatively and qualitatively detect HCV are equally important for a variety of clinical applications including diagnostic confirmation, blood bank screening, and monitoring of therapeutic efficacy. Sensitivity, accuracy, and dynamic range of HCV viral load tests are critical parameters for these applications.

HCV is classified in six major genotypes (numbered 1-6) and approximately 80 or more subtypes. In patients with confirmed HCV infection, genotype testing is often recommended. HCV genotype testing is used to determine the required length and potential response to an anti-viral therapy. The HCV genome exhibits significant difference among the various genotypes and subtypes. Approximately 60-80% homology is observed between genotypes and approximately 95% homology is observed within members of the same viral genotype (i.e. between subtypes). Even greater homology is found within the 5′ and 3′ non-coding (NC) regions. These regions show the most conserved sequences among all HCV genotypes and subtypes.

Currently, all commercially available HCV viral load assays target the conserved 5′ non-coding (5′ NC) region of the HCV genome. The present inventors have found that the 3′ NC region may be useful for HCV detection. Like the 5′ NC region, the 98 nucleotides at the extreme 3′ end of the HCV genome is highly conserved region among all six HCV genotypes. However, other methods of detection (e.g. TaqMan®, molecular beacons, etc.) have unexpectedly proven inadequate for sensitive and robust detection of the 3′ NC of HCV. While not wishing to be bound by theory, the inventors believe that performance of these technologies is hindered because the assays technically require the use of probes, which have difficulty annealing due to the secondary structure of the 3′ NC region. In contrast, various embodiments of the present invention require no probes, and can reliably amplify an amplicon from the 3′ NC region.

For example, in one embodiment, the 3′ NC region of the HCV genome can be targeted using a novel quantitative real-time PCR assay. This assay uses a Dabcyl quencher covalently attached to iso-G triphosphate, which is site-specifically incorporated opposite an iso-C base, adjacent to a 5′ fluorescent label in one of the primers. The methods do not require probes because specific amplification during PCR can be measured by a decrease in fluorescence. The lack of probes promotes higher sensitivity and a broader linear dynamic range for virtually any qualitative or quantitative assay.

The 3′ NC region of HCV has only 98 nucleotides, and comprises the sequence according to:

(SEQ ID NO: 1)

UGGUGGCUCCAUCUUAGCCCUAGUCACGGCUAGCUGUGAAAGGUCCGUGA
GCGGCAUGACUGCAGAGAGUGCUGAUACUGGCCUCUCUGCAGAUCAUGU

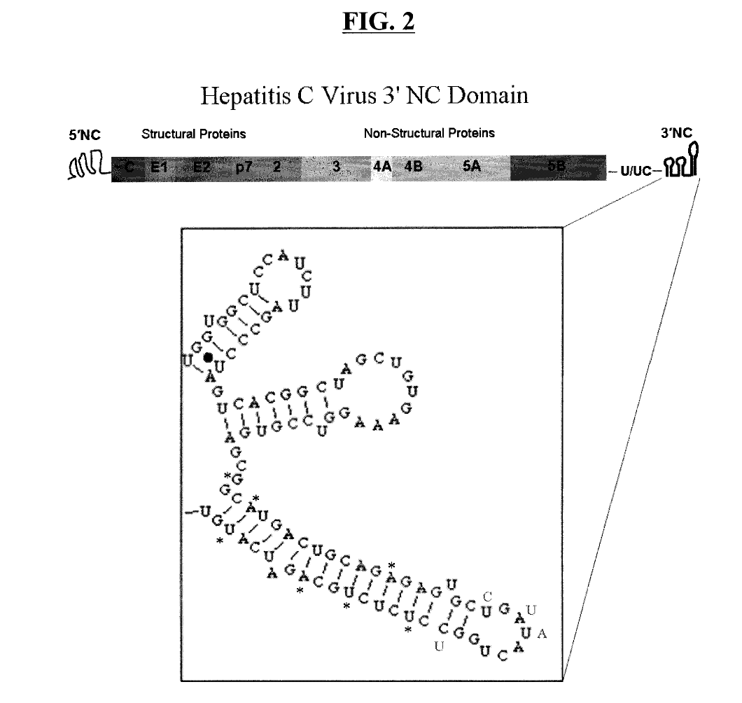

As shown in FIG. 2, the majority of the bases in the 3′ NC region form secondary structures. In general, the 3′ NC sequence is highly conserved among genotypes. Polymorphisms differing from genotype 1 have been identified across the six genotypes, their positions indicated by an asterisk (*) in FIG. 2. In one embodiment, primers capable of amplifying all six HCV genotypes are used. In other embodiments, known polymorphisms can be used to design specific primers (as described below) to one or more genotypes.

The present invention is described herein using several definitions, as set forth below and throughout the specification.

As used herein, unless otherwise stated, the singular forms “a,” “an,” and “the” include plural reference. Thus, for example, a reference to “an oligonucleotide” includes a plurality of oligonucleotide molecules, and a reference to “a nucleic acid” is a reference to one or more nucleic acids.

As used herein, “nucleic acid,” “nucleotide sequence,” or “nucleic acid sequence” refer to a nucleotide, oligonucleotide, polynucleotide, or any fragment thereof and to naturally occurring or synthetic molecules. These phrases also refer to DNA or RNA of genomic or synthetic origin which may be single-stranded or double-stranded and may represent the sense or the antisense strand, or to any DNA-like or RNA-like material. An “RNA equivalent,” in reference to a DNA sequence, is composed of the same linear sequence of nucleotides as the reference DNA sequence with the exception that all occurrences of the nitrogenous base thymine are replaced with uracil, and the sugar backbone is composed of ribose instead of deoxyribose. RNA may be used in the methods described herein and/or may be converted to cDNA by reverse-transcription for use in the methods described herein.

As used herein, “HCV nucleic acid” refers to a nucleic acid or nucleic acid fragment from the HCV genome (RNA or DNA), wherein the HCV nucleic acid is suspected in a sample and will be detected or quantified in a method or system as disclosed herein. HCV nucleic acid contains the target nucleic acid sequences that are actually assayed during an assay procedure. In at least some embodiments, the HCV nucleic acid, if present in the sample, is used as a template for amplification according to the methods disclosed herein.

The term also includes nucleic acid fragments of the HCV genome and may not necessarily comprise the full length sequence. Thus, a “fragment” means any contiguous portion or amount less than the whole. Typically, a fragment of a nucleic acid sequence is capable of being amplified in a PCR reaction and includes contiguous sequences of at least 15, at least 16, at least 17, at least 18, at least 19, or at least 20 consecutive nucleotides from the nucleic acid sequence, e.g., the HCV genome or SEQ ID NO:1. In some embodiments, the one or more contiguous sequences have at least 25, at least 30, at least 40, or at least 50 or 100 consecutive nucleotides from the nucleic acid sequence. In some embodiments of the present methods, the amplification product is a fragment comprising or consisting of nucleotides 5 to 52 of SEQ ID NO:1. Such fragments may also include polymorphisms such that the fragment has at least 80%, at least 85%, at least 90%, or at least 95% sequence identity to the same fragment of the nucleic acid without any polymorphisms. In some embodiments, the fragment has 1, 2, 3, 4, 5, 6, 7, 8, 9, or 10 nucleotide polymorphisms in the sequence of SEQ ID NO: 1.

As used herein, the term “sample” is used in its broadest sense. A sample may include a bodily tissue or a bodily fluid including but not limited to blood (or a fraction of blood such as plasma or serum), lymph, mucus, tears, urine, and saliva. A sample may include an extract from a cell, a chromosome, organelle, or a virus. A sample may be a “cell-free” sample, meaning that the volume of cells in the sample are less than about 2% of the total sample volume (preferably less than about 1% of the total sample volume). A sample may comprise DNA (e.g., genomic DNA), RNA (e.g., mRNA), and cDNA, any of which may be amplified to provide amplified nucleic acid. For example, a sample may include nucleic acid in solution or bound to a substrate (e.g., as part of a microarray). A sample may be obtained from any patient. In particular, a sample may be obtained from a patient having or suspected to be at risk for HCV infection.

As used herein, an oligonucleotide is understood to be a molecule that has a sequence of bases on a backbone comprised mainly of identical monomer units at defined intervals. The bases are arranged on the backbone in such a way that they can enter into a bond with a nucleic acid having a sequence of bases that are complementary to the bases of the oligonucleotide. The most common oligonucleotides have a backbone of sugar phosphate units. A distinction may be made between oligodeoxyribonucleotides, which do not have a hydroxyl group at the 2′ position, and oligoribonucleotides, which have a hydroxyl group in this position. Oligonucleotides also may include derivatives, in which the hydrogen of the hydroxyl group is replaced with organic groups, e.g., an allyl group.

An oligonucleotide is a nucleic acid that includes at least two nucleotides. Oligonucleotides used in the methods disclosed herein typically include at least about ten (10) nucleotides and more typically at least about fifteen (15) nucleotides. Preferred oligonucleotides for the methods disclosed herein include about 10-25 nucleotides.

An oligonucleotide may be designed to function as a “primer.” A “primer” is a short nucleic acid, usually a single strand DNA oligonucleotide, which may be annealed to a target polynucleotide by complementary base-pairing. The primer may then be extended along the target DNA strand by a DNA polymerase enzyme. If the target is RNA, a primer may anneal and then be extended by a reverse transcriptase enzyme. Primer pairs can be used for amplification (and identification) of a nucleic acid sequence (e.g., by the polymerase chain reaction (PCR)). An oligonucleotide may be designed to function as a “probe.”

A nucleic acid sequence may be “complementary” to a target nucleic acid sequence. As used herein, the terms “complementary” or “complementarity,” when used in reference to nucleic acids (i.e., a sequence of nucleotides such as an oligonucleotide or a target nucleic acid), refer to sequences that are related by base-pairing rules. For natural bases, the base pairing rules are those developed by Watson and Crick. For non-natural bases, as described herein, the base-pairing rules include the formation of hydrogen bonds in a manner similar to the Watson-Crick base pairing rules or by hydrophobic, entropic, or van der Waals forces. As an example, for the sequence “T-G-A”, the complementary sequence is “A-C-T.” Complementarity can be “partial,” in which only some of the bases of the nucleic acids are matched according to the base pairing rules. Alternatively, there can be “complete” or “total” complementarity between the nucleic acids. The degree of complementarity between the nucleic acid strands has effects on the efficiency and strength of hybridization between the nucleic acid strands.

Oligonucleotides as described herein typically are capable of forming hydrogen bonds with oligonucleotides having a complementary base sequence. These bases may include the natural bases such as A, G, C, T and U, as well as non-natural, such as iso-C or iso-G. As described herein, a first sequence of an oligonucleotide is described as being 100% complementary with a second sequence of an oligonucleotide when the consecutive bases of the first sequence (read 5′ to 3′) follow the Watson-Crick rule of base pairing as compared to the consecutive bases of the second sequence (read 3′ to 5′). An oligonucleotide may include nucleotide substitutions. For example, an artificial base may be used in place of a natural base such that the artificial base exhibits a specific interaction that is similar to the natural base.

An oligonucleotide that is specific for a target nucleic acid also may be specific for a nucleic acid sequence that has “homology” to the target nucleic acid sequence. As used herein, “homology” refers to sequence similarity or, interchangeably, sequence identity, between two or more polynucleotide sequences or two or more polypeptide sequences. The terms “percent identity” and “% identity” as applied to polynucleotide sequences, refer to the percentage of residue matches between at least two polynucleotide sequences aligned using a standardized algorithm (e.g., BLAST).

An oligonucleotide that is specific for a target nucleic acid will “hybridize” to the target nucleic acid under suitable conditions. As used herein, “hybridization” or “hybridizing” refers to the process by which a oligonucleotide single strand anneals with a complementary strand through base pairing under defined hybridization conditions. “Specific hybridization” is an indication that two nucleic acid sequences share a high degree of complementarity. Specific hybridization complexes form under permissive annealing conditions and remain hybridized after any subsequent washing steps. “Hybridizing” sequences which bind under conditions of low stringency are those which bind under non-stringent conditions (6×SSC/50% formamide at room temperature) and remain bound when washed under conditions of low stringency (2×SSC, 42° C.). Hybridizing under high stringency refers to the above conditions in which washing is performed at 2×SSC, 65° C. (where SSC is 0.15M NaCl, 0.015M sodium citrate, pH 7.2). Oligonucleotides used as specific primers for amplifying a target nucleic acid generally are capable of specifically hybridizing to the target nucleic acid.

As used herein, “amplification” or “amplifying” refers to the production of additional copies of a nucleic acid sequence. Amplification is generally carried out using polymerase chain reaction (PCR) technologies known in the art. The term “amplification reaction system” refers to any in vitro means for multiplying the copies of a target sequence of nucleic acid. The term “amplification reaction mixture” refers to an aqueous solution comprising the various reagents used to amplify a target nucleic acid. These may include enzymes (e.g., a thermostable polymerase), aqueous buffers, salts, amplification primers, target nucleic acid, and nucleoside triphosphates, and optionally at least one labeled probe and/or optionally at least one agent for determining the melting temperature of an amplified target nucleic acid (e.g., a fluorescent intercalating agent that exhibits a change in fluorescence in the presence of double-stranded nucleic acid). Optionally, the amplification mixture may include a reverse transcriptase enzyme used to make a cDNA copy of RNA.

The amplification methods described herein may include “real-time monitoring” or “continuous monitoring.” These terms refer to monitoring multiple times during a cycle of PCR, preferably during temperature transitions, and more preferably obtaining at least one data point in each temperature transition. For example, the data point may comprise a measurement of the fluorescence emitted from the sample. The term “homogeneous detection assay” is used to describe an assay that includes coupled amplification and detection, which may include “real-time monitoring” or “continuous monitoring.”

Amplification of nucleic acids may include amplification of nucleic acids or subregions or fragments of these nucleic acids. For example, amplification may include amplifying portions of nucleic acids between 50 and 300 bases long by selecting the proper primer sequences and using PCR.

The disclosed methods may include amplifying at least one nucleic acid in the sample. In the disclosed methods, amplification may be monitored using real-time methods. Amplification mixtures may include natural nucleotides (e.g., nucleotides having A, C, G, T, and U as a nucleobase) and non-natural nucleotides (i.e., nucleotides having other than A, C, G, T, and U as a nucleobase, e.g., nucleotides that include iso-C and iso-G as a nucleobase). Non-natural nucleotides and bases are described in U.S. patent application publication 2002-0150900, which is incorporated herein by reference in its entirety. The nucleotides, which may include non-natural nucleotides may include a label (e.g., a quencher or a fluorophore). The methods may include amplifying multiple nucleic acids in sample, also known as “multiplex detection” or “multiplexing.” As used herein the term “multiplex PCR” refers to PCR, which involves adding more than one set of PCR primers to the reaction in order to detect and quantify multiple nucleic acids, including nucleic acids from one or more pathogens. Furthermore, multiplexing with an internal control (e.g., 18s rRNA, GAPDH, or β-actin) provides a control for the PCR amplification.

The oligonucleotides of the present methods may function as primers for PCR or reverse transcription. In some embodiments, the oligonucleotides are labeled. For example, the oligonucleotides may be labeled with a reporter that emits a detectable signal (e.g., a fluorophore). The oligonucleotides may include at least one non-natural nucleotide. For example, the oligonucleotides may include at least one nucleotide that includes a nucleobase other than A, C, G, T, or U (e.g., iso-C or iso-G). Where the oligonucleotide is used as a primer for PCR, the amplification mixture may include at least one nucleotide that is labeled with a quencher (e.g., Dabcyl). The labeled nucleotide may include at least one non-natural nucleotide. For example, the labeled nucleotide may include at least one nucleobase that is not A, C, G, T, or U (e.g., iso-C or iso-G).

In some embodiments, the oligonucleotide may be designed not to form an intramolecular structure such as a hairpin. In other embodiments, the oligonucleotide may be designed to form an intramolecular structure such as a hairpin. For example, the oligonucleotide may be designed to form a hairpin structure that is altered after the oligonucleotide hybridizes to a target nucleic acid, and optionally, after the target nucleic acid is amplified using the oligonucleotide as a primer.

The oligonucleotide may be labeled with a fluorophore that exhibits quenching when incorporated in an amplified product as a primer. In other embodiments, the oligonucleotide may emit a detectable signal after the oligonucleotide is incorporated in an amplified product as a primer (e.g., inherently, or by fluorescence induction or fluorescence dequenching). Such primers are known in the art (e.g., LightCycler primers, Amplifluor® Primers, Scorpion® Primers and Lux™ Primers). The fluorophore used to label the oligonucleotide may emit a signal when intercalated in double-stranded nucleic acid. As such, the fluorophore may emit a signal after the oligonucleotide is used as a primer for amplifying the nucleic acid.

The oligonucleotides that are used in the disclosed methods may be suitable as primers for amplifying at least one nucleic acid in the sample and as probes for detecting at least one nucleic acid in the sample. In some embodiments, the oligonucleotides are labeled with at least one fluorescent dye, which may produce a detectable signal. The fluorescent dye may function as a fluorescence donor for fluorescence resonance energy transfer (FRET). The detectable signal may be quenched when the oligonucleotide is used to amplify a target nucleic acid. For example, the amplification mixture may include nucleotides that are labeled with a quencher for the detectable signal emitted by the fluorophore. Optionally, the oligonucleotides may be labeled with a second fluorescent dye or a quencher dye that may function as a fluorescence acceptor (e.g., for FRET). Where the oligonucleotide is labeled with a first fluorescent dye and a second fluorescent dye, a signal may be detected from the first fluorescent dye, the second fluorescent dye, or both.

The disclosed methods may be performed with any suitable number of oligonucleotides. Where a plurality of oligonucleotides are used (e.g., two or more oligonucleotides), different oligonucleotide may be labeled with different fluorescent dyes capable of producing a detectable signal. In some embodiments, oligonucleotides are labeled with at least one of two different fluorescent dyes. In further embodiments, oligonucleotides are labeled with at least one of three different fluorescent dyes.

In some embodiments, each different fluorescent dye emits a signal that can be distinguished from a signal emitted by any other of the different fluorescent dyes that are used to label the oligonucleotides. For example, the different fluorescent dyes may have wavelength emission maximums all of which differ from each other by at least about 5 nm (preferably by least about 10 nm). In some embodiments, each different fluorescent dye is excited by different wavelength energies. For example, the different fluorescent dyes may have wavelength absorption maximums all of which differ from each other by at least about 5 nm (preferably by at least about 10 nm).

Where a fluorescent dye is used to determine the melting temperature of a nucleic acid in the method, the fluorescent dye may emit a signal that can be distinguished from a signal emitted by any other of the different fluorescent dyes that are used to label the oligonucleotides. For example, the fluorescent dye for determining the melting temperature of a nucleic acid may have a wavelength emission maximum that differs from the wavelength emission maximum of any other fluorescent dye that is used for labeling an oligonucleotide by at least about 5 nm (preferably by least about 10 nm). In some embodiments, the fluorescent dye for determining the melting temperature of a nucleic acid may be excited by different wavelength energy than any other of the different fluorescent dyes that are used to label the oligonucleotides. For example, the fluorescent dye for determining the melting temperature of a nucleic acid may have a wavelength absorption maximum that differs from the wavelength absorption maximum of any fluorescent dye that is used for labeling an oligonucleotide by at least about 5 nm (preferably by least about 10 nm).

The methods may include determining the melting temperature of at least one nucleic acid in a sample (e.g., “amplified nucleic acid” otherwise called “an amplicon”), which may be used to identify the nucleic acid. This “end-point analysis” or “melting curve analysis” involves determining the melting temperature by exposing an amplicon to a temperature gradient and observing a detectable signal from a fluorophore. Optionally, where the oligonucleotides of the method are labeled with a first fluorescent dye, determining the melting temperature of the detected nucleic acid may include observing a signal from a second fluorescent dye that is different from the first fluorescent dye. In some embodiments, the second fluorescent dye for determining the melting temperature of the detected nucleic acid is an intercalating agent. Suitable intercalating agents may include, but are not limited to SYBR™ Green 1 dye, SYBR dyes, Pico Green, SYTO dyes, SYTOX dyes, ethidium bromide, ethidium homodimer-1, ethidium homodimer-2, ethidium derivatives, acridine, acridine orange, acridine derivatives, ethidium-acridine heterodimer, ethidium monoazide, propidium iodide, cyanine monomers, 7-aminoactinomycin D, YOYO-1, TOTO-1, YOYO-3, TOTO-3, POPO-1, BOBO-1, POPO-3, BOBO-3, LOLO-1, JOJO-1, cyanine dimers, YO-PRO-1, TO-PRO-1, YO-PRO-3, TO-PRO-3, TO-PRO-5, PO-PRO-1, BO-PRO-1, PO-PRO-3, BO-PRO-3, LO-PRO-1, JO-PRO-1, and mixture thereof. In suitable embodiments, the selected intercalating agent is SYBR™ Green 1 dye.

Typically, an intercalating agent used in the method will exhibit a change in fluorescence when intercalated in double-stranded nucleic acid. A change in fluorescence may include an increase in fluorescence intensity or a decrease in fluorescence intensity. For example, the intercalating agent may exhibit a increase in fluorescence when intercalated in double-stranded nucleic acid, and a decrease in fluorescence when the double-stranded nucleic acid is melted. A change in fluorescence may include a shift in fluorescence spectra (i.e., a shift to the left or a shift to the right in maximum absorbance wavelength or maximum emission wavelength). For example, the intercalating agent may emit a fluorescent signal of a first wavelength (e.g., green) when intercalated in double-stranded nucleic and emit a fluorescent signal of a second wavelength (e.g., red) when not intercalated in double-stranded nucleic acid. A change in fluorescence of an intercalating agent may be monitored at a gradient of temperatures to determine the melting temperature of the nucleic acid (where the intercalating agent exhibits a change in fluorescence when the nucleic acid melts).

In the disclosed methods, each of these amplified target nucleic acids may have different melting temperatures. For example, each of these amplified target nucleic acids may have a melting temperature that differs by at least about 1° C., more preferably by at least about 2° C., or even more preferably by at least about 4° C. from the melting temperature of any of the other amplified target nucleic acids.

The methods disclosed herein may include transcription of RNA to DNA (i.e., reverse transcription). For example, reverse transcription may be performed prior to amplification by the addition of a reverse transcriptase enzyme, such as that from Moloney murine leukemia virus (MMLV) or avian myeloblastosis virus (AMV).

As used herein, “labels” or “reporter molecules” are chemical or biochemical moieties useful for labeling a nucleic acid (including a single nucleotide), amino acid, or antibody. “Labels” and “reporter molecules” include fluorescent agents, chemiluminescent agents, chromogenic agents, quenching agents, radionucleotides, enzymes, substrates, cofactors, inhibitors, magnetic particles, and other moieties known in the art. “Labels” or “reporter molecules” are capable of generating a measurable signal and may be covalently or noncovalently joined to an oligonucleotide or nucleotide (e.g., a non-natural nucleotide).

The oligonucleotides and nucleotides (e.g., non-natural nucleotides) of the disclosed methods may be labeled with a “fluorescent dye” or a “fluorophore.” As used herein, a “fluorescent dye” or a “fluorophore” is a chemical group that can be excited by light to emit fluorescence. Some suitable fluorophores may be excited by light to emit phosphorescence. Dyes may include acceptor dyes that are capable of quenching a fluorescent signal from a fluorescent donor dye. Dyes that may be used in the disclosed methods include, but are not limited to, the following dyes and/or dyes sold under the following tradenames: 1,5 IAEDANS; 1,8-ANS; 4-Methylumbelliferone; 5-carboxy-2,7-dichlorofluorescein; 5-Carboxyfluorescein (5-FAM); 5-Carboxytetramethylrhodamine (5-TAMRA); 5-Hydroxy Tryptamine (HAT); 5-ROX (carboxy-X-rhodamine); 6-Carboxyrhodamine 6G; 6-JOE; 7-Amino-4-methylcoumarin; 7-Aminoactinomycin D (7-AAD); 7-Hydroxy-4-methylcoumarin; 9-Amino-6-chloro-2-methoxyacridine; ABQ; Acid Fuchsin; ACMA (9-Amino-6-chloro-2-methoxyacridine); Acridine Orange; Acridine Red; Acridine Yellow; Acriflavin; Acriflavin Feulgen SITSA; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC; AMCA-S; AMCA (Aminomethylcoumarin); AMCA-X; Aminoactinomycin D; Aminocoumarin; Aminomethylcoumarin (AMCA); Anilin Blue; Anthrocyl stearate; APC (Allophycocyanin); APC-Cy7; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); Berberine Sulphate; Beta Lactamase; BFP blue shifted GFP (Y66H); Blue Fluorescent Protein; BFP/GFP FRET; Bimane; Bisbenzamide; Bisbenzimide (Hoechst); Blancophor FFG; Blancophor SV; BOBO™-1; BOBO™-3; Bodipy 492/515; Bodipy 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy FL; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; Calcein; Calcein Blue; Calcium Crimson™; Calcium Green; Calcium Orange; Calcofluor White; Cascade Blue™; Cascade Yellow; Catecholamine; CCF2 (GeneBlazer); CFDA; CFP—Cyan Fluorescent Protein; CFP/YFP FRET; Chlorophyll; Chromomycin A; CL-NERF (Ratio Dye, pH); CMFDA; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM Methylcoumarin; CTC; CTC Formazan; Cy2™; Cy3.1 8; Cy3.5™; Cy3™; Cy5.1 8; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3; DCFDA; DCFH (Dichlorodihydrofluorescein Diacetate); DDAO; DHR (Dihydrorhodamine 123); Di-4-ANEPPS; Di-8-ANEPPS (non-ratio); DiA (4-Di-16-ASP); Dichlorodihydrofluorescein Diacetate (DCFH); DiD—Lipophilic Tracer; DiD (DiIC18(5)); DIDS; Dihydrorhodamine 123 (DHR); DiI (DiIC18(3)); Dinitrophenol; DiO (DiOC18(3)); DiR; DiR (DiIC18(7)); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (III) chloride; EYFP; Fast Blue; FDA; Feulgen (Pararosaniline); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; Fluor X; FM 1-43™; FM 4-46; Fura Red™; Fura Red™/Fluo-3; Fura-2; Fura-2/BCECF; Genacryl Brilliant Red B; Genacryl Brilliant Yellow 10GF; Genacryl Pink 3G; Genacryl Yellow 5GF; GeneBlazer (CCF2); GFP (S65T); GFP red shifted (rsGFP); GFP wild type, non-UV excitation (wtGFP); GFP wild type, UV excitation (wtGFP); GFPuv; Gloxalic Acid; Granular Blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1; Indodicarbocyanine (DiD); Indotricarbocyanine (DiR); Intrawhite Cf; JC-1; JO-JO-1; JO-PRO-1; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-Indo-1; Magnesium Green; Magnesium Orange; Malachite Green; Marina Blue; Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; NED™; Nitrobenzoxadidole; Noradrenaline; Nuclear Fast Red; Nuclear Yellow; Nylosan Brilliant lavin E8G; Oregon Green; Oregon Green 488-X; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed [Red 613]; Phloxin B (Magdala Red); Phorwite AR; Phorwite BKL; Phorwite Rev; Phorwite RPA; Phosphine 3R; Phycoerythrin B [PE]; Phycoerythrin R [PE]; PKH26 (Sigma); PKH67; PMIA; Pontochrome Blue Black; POPO-1; POPO-3; PO-PRO-1; PO-PRO-3; Primuline; Procion Yellow; Propidium Iodid (PI); PyMPO; Pyrene; Pyronine; Pyronine B; Pyrozal Brilliant Flavin 7GF; QSY 7; Quinacrine Mustard; Red 613 [PE-TexasRed]; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine Phalloidine; Rhodamine Red; Rhodamine WT; Rose Bengal; R-phycocyanine; R-phycoerythrin (PE); RsGFP; S65A; S65C; S65L; S65T; Sapphire GFP; SBFI; Serotonin; Sevron Brilliant Red 2B; Sevron Brilliant Red 4G; Sevron Brilliant Red B; Sevron Orange; Sevron Yellow L; sgBFP™; sgBFP™ (super glow BFP); sgGFP™; sgGFP™ (super glow GFP); SITS; SITS (Primuline); SITS (Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3-sulfopropyl)quinolinium); Stilbene; Sulphorhodamine B can C; Sulphorhodamine G Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; TET™; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TRITC TetramethylRodaminelsoThioCyanate; True Blue; TruRed; Ultralite; Uranine B; Uvitex SFC; VIC®; wt GFP; WW 781; X-Rhodamine; XRITC; Xylene Orange; Y66F; Y66H; Y66W; Yellow GFP; YFP; YO-PRO-1; YO-PRO-3; YOYO-1; YOYO-3; and salts thereof.

Fluorescent dyes or fluorophores may include derivatives that have been modified to facilitate conjugation to another reactive molecule. As such, fluorescent dyes or fluorophores may include amine-reactive derivatives such as isothiocyanate derivatives and/or succinimidyl ester derivatives of the fluorophore.

The oligonucleotides and nucleotides of the disclosed methods (e.g., non-natural nucleotides) may be labeled with a quencher. Quenching may include dynamic quenching (e.g., by FRET), static quenching, or both. Suitable quenchers may include Dabcyl. Suitable quenchers may also include dark quenchers, which may include black hole quenchers sold under the tradename “BHQ” (e.g., BHQ-0, BHQ-1, BHQ-2, and BHQ-3, Biosearch Technologies, Novato, Calif.). Dark quenchers also may include quenchers sold under the tradename “QXL™” (Anaspec, San Jose, Calif.). Dark quenchers also may include DNP-type non-fluorophores that include a 2,4-dinitrophenyl group.

The oligonucleotides or nucleotides (e.g., non-natural nucleotides) of the present methods may be labeled with a donor fluorophore and an acceptor fluorophore (or quencher dye) that are present in the oligonucleotides at positions that are suitable to permit FRET (or quenching). Labeled oligonucleotides that are suitable for the present methods may include but are not limited to oligonucleotides designed to function as LightCycler primers or probes, Taqman® Probes, Molecular Beacon Probes, Amplifluor® Primers, Scorpion® Primers, and Lux™ Primers.

In some embodiments, the disclosed assays are used for the detection of HCV nucleic acid. The assays may be performed using real-time or continuous methods using any suitable commercial instruments. The disclosed technology may be used to detect nucleic acid targets obtained from any source (e.g., human, animal and infectious disease samples). Advantages of the assays disclosed herein may include: high sensitivity, high specificity, rapid cycling with real-time readout, thermal melt at the end of run to verify specific amplification of target sequences, inclusion of internal RT-PCR control, excellent stability, rapid creation of new assays based on genetic sequences, rapid reaction time, and multiplexing capabilities by using dyes or melt-curve analyses.

The methods disclosed herein may use PCR to amplify nucleic acids from the target and/or non-target species. The methods disclosed herein may utilize two or more specific primers, a universal primer, and, typically a non-natural nucleotide base and nucleic acid polymerase. Other assay methods using non-natural nucleotide bases are described in U.S. Patent Application Publication 2002-0150900.

As used herein, “target species” or “target subspecies” refers to a species or subspecies of an organism, in or suspected to be in a sample. The target species or subspecies contains a target nucleic acid that may be used to distinguish it from non-target species or subspecies.

As used herein, “non-target species” or “non-target subspecies” refers to one or more species or subspecies of an organism different from the target species or subspecies. A non-target species or subspecies is distinguished from the target species or subspecies by containing a nucleic acid that is similar but not identical to the target nucleic acid of the target species or subspecies.

As used herein, “target nucleic acid” refers to a nucleic acid containing a nucleic acid sequence, suspected to be in a sample and to be detected or quantified in a method or system as disclosed herein. Target nucleic acids contain the target nucleic acid sequences that are actually assayed during an assay procedure. The target can be directly or indirectly assayed. In at least some embodiments, the target nucleic acid, if present in the sample, is used as a template for amplification according to the methods disclosed herein. Target nucleic acid may include HCV nucleic acid.

As used herein, the term “thermostable nucleic acid polymerase” refers to an enzyme that catalyzes the polymerization of nucleosides and which is relatively stable to heat when compared, for example, to nucleotide polymerases from E. coli. Generally, the enzyme will initiate synthesis at the 3′-end of the primer annealed to the target sequence, and will proceed in the 5′-direction along the template, and if possessing a 5′ to 3′ nuclease activity, hydrolyzing an intervening, annealed oligonucleotide to release intervening nucleotide bases or oligonucleotide fragments, until synthesis terminates. A thermostable enzyme has activity at a temperature of at least about 37° C. to about 42° C., typically in the range from about 50° C. to about 75° C. Representative thermostable polymerases include, for example, thermostable polymerases such as native and altered polymerases of Thermus species, including, but not limited to, Thermus aquaticus (Taq), Thermus flavus (Tfl), and Thermus thermophilus (Tth), and of the Thermotoga species, including, but not limited to, Thermotoga neapolitana.

As used herein, the term “mutant” or “polymorphism” refers to the condition in which two or more different nucleotide sequences can exist at a particular site in DNA and includes any nucleotide variation, such as single or multiple nucleotide substitutions, deletions or insertions. These nucleotide variations can be species specific, mutant or polymorphic variations (e.g., allele specific variations). At least some of the embodiments of the methods described herein can detect single nucleotide changes in nucleic acids, additions or deletions, as well as multiple-base variations. In addition, the process herein can detect polymorphisms, which are not necessarily associated with a disease, but are merely a condition in which two or more different nucleotide sequences (whether having substituted, deleted or inserted nucleotide base pairs) can exist at a particular site in the nucleic acid in the population. For example, polymorphisms existing between genotypes may be detected in accordance with the present invention.

As used herein, “specifically hybridizing” or “specifically hybridize” refer to conditions under which an oligonucleotide binds to a target nucleic acid, but to a minimal number of other sequences. Specific hybridization is typically assessed under stringent conditions. For example, a “specific primer” may specifically hybridize to a target nucleic acid because the specific primer is complementary to the target nucleic acid. “Complementarity” may include full or complete complementarity. However, a primer may be specific for a target nucleic acid without being fully or completely complementary. A primer may specifically hybridize to a target acid under suitable conditions (e.g., under conditions which A-T and G-C base pairing occurs). A primer may specifically hybridize to a target acid under suitable conditions that include stringent conditions.

As used herein, “universal primer” refers to a primer that can specifically hybridize to two or more different target nucleic acids in a sample (e.g., 3 or more, 4 or more, 5 or more, 10 or more, 15 or more, or 25 or more different target nucleic acids in a sample). A “universal primer” may hybridize to a region of the different target nucleic acids that is identical or that has substantial identity to provide for specific hybridization of the “universal primer” to the different target nucleic acids. A “universal primer” may be complementary to a nucleic acid sequence that is common to all the genotypes and subtypes or selected genotypes and subtypes of HCV nucleic acid in a sample.

In some embodiments of the methods and kits disclosed herein, a sample is suspected to contain a target species or subspecies, e.g. an HCV nucleic acid. A first specific primer for the target nucleic acid and a second specific primer are added to the sample along with a non-natural nucleotide conjugated to a label. Each specific primer may include an identical non-natural nucleotide base and a label (e.g., a fluorescent label, a radiolabel, and an enzyme label). Each label may be different from the other. For example, one label may be fluorescein (FAM) and the other label may be hexachlorofluorescein (HEX). Typically, the specific primers are tailed primers (i.e., primers with non-complementary nucleotides added to one end of the primer). The tails may be added to the 5′ end of the primers. The tails may be designed to improve the specificity of the primers by reducing mispriming during PCR. For example, the tail sequences can be designed to add about 10° C. to the Tₘ of the specific primers (e.g., to reduce “mispriming”).

In some embodiments, the specific primers are allowed to anneal to the nucleic acids from the target species or subspecies. PCR using a nucleic acid polymerase (as herein described) is performed with chain extension of the annealed specific primer to form a double stranded product. One of the two strands of the product may incorporate a non-natural nucleotide base bearing a fluorescent quencher such as dabcyl. PCR is run for the desired number of cycles to obtain the double-stranded amplification product. As more of the double stranded amplification product accumulates having both a fluorophore and a fluorescent quencher, the fluorescent signal from the specific primer(s) being incorporated into the amplified product will decrease.

As will be apparent from the discussion herein, the relative sizes of the specific primers, as well as the amplified portion of the target and non-target nucleic acids, will vary depending upon the particular application. Further, the relative location of the primers along the target nucleic acid will vary. Additionally, the location of the non-natural base and labels used in the methods disclosed herein will vary depending upon application.

### Polymerases

Disclosed herein are methods that may utilize the polymerase chain reaction, to detect nucleic acids of interest in a sample (i.e., nucleic acids of the target and non-target species or subspecies). Suitable nucleic acid polymerases include, for example, polymerases capable of extending an oligonucleotide by incorporating nucleic acids complementary to a template oligonucleotide. For example, the polymerase can be a DNA polymerase.

Enzymes having polymerase activity catalyze the formation of a bond between the 3′ hydroxyl group at the growing end of a nucleic acid primer and the 5′ phosphate group of a nucleotide triphosphate. These nucleotide triphosphates are usually selected from deoxyadenosine triphosphate (A), deoxythymidine triphosphate (T), deoxycytosine triphosphate (C) and deoxyguanosine triphosphate (G). However, in at least some embodiments, polymerases useful for the methods disclosed herein also may incorporate non-natural bases using nucleotide triphosphates of those non-natural bases.

Because the relatively high temperatures necessary for strand denaturation during methods such as PCR can result in the irreversible inactivation of many nucleic acid polymerases, nucleic acid polymerase enzymes useful for performing the methods disclosed herein preferably retain sufficient polymerase activity to complete the reaction when subjected to the temperature extremes of methods such as PCR. In suitable embodiments, the nucleic acid polymerase enzymes useful for the methods disclosed herein are thermostable nucleic acid polymerases. Suitable thermostable nucleic acid polymerases include, but are not limited to, enzymes derived from thermophilic organisms. Examples of thermophilic organisms from which suitable thermostable nucleic acid polymerase can be derived include, but are not limited to, Thermus aquaticus, Thermus thermophilus, Thermus flavus, Thermotoga neapolitana and species of the Bacillus, Thermococcus, Sulfobus, and Pyrococcus genera. Suitable thermostable nucleic acid polymerases, such as those described above, are commercially available.

A number of nucleic acid polymerases possess activities in addition to nucleic acid polymerase activity; these can include 5′ to 3′ exonuclease activity and 3′ to 5′ exonuclease activity. The 5′ to 3′ and 3′ to 5′ exonuclease activities are known to those of ordinary skill in the art. The 3′ to 5′ exonuclease activity improves the accuracy of the newly-synthesized strand by removing incorrect bases that have been incorporated. In contrast, the 5′ to 3′ exonuclease activity often present in nucleic acid polymerase enzymes can be undesirable in a particular application since it may digest nucleic acids, including primers, that have an unprotected 5′ end. Thus, a thermostable nucleic acid polymerase with an attenuated 5′ to 3′ exonuclease activity, or in which such activity is absent, is a desired characteristic of an enzyme for use in at least some embodiments of the methods disclosed herein. In other embodiments, the polymerase is desired to have 5′ to 3′ exonuclease activity to efficiently cleave the reporter and release labeled fragments so that the signal is directly or indirectly generated.

Suitable nucleic acid polymerases having no 5′ to 3′ exonuclease activity or an attenuated 5′ to 3′ exonuclease activity are known in the art. Various nucleic acid polymerase enzymes have been described where a modification has been introduced in a nucleic acid polymerase which accomplishes this object. For example, the Klenow fragment of E. coli DNA polymerase I can be produced as a proteolytic fragment of the holoenzyme in which the domain of the protein controlling the 5′ to 3′ exonuclease activity has been removed. Suitable nucleic acid polymerases deficient in 5′ to 3′ exonuclease activity are commercially available. Examples of commercially available polymerases that are deficient in 5′ to 3′ exonuclease activity include AMPLITAQ STOFFEL™ DNA polymerase and KlenTaq™ DNA polymerase.

As an alternative to using a single polymerase, any of the methods described herein can be performed using multiple enzymes. For example, a polymerase, such as an exo-nuclease deficient polymerase, and an exo-nuclease can be used in combination. Another example is the use of an exo-nuclease deficient polymerase and a thermostable flap endonuclease. In addition, it will be recognized that RNA can be used as a sample and that a reverse transcriptase can be used to transcribe the RNA to cDNA. The transcription can occur prior to or during PCR amplification.

### Specific Primers

Disclosed herein are methods for detecting a target nucleic acid that may utilize PCR. The methods may involve a polymerase, a first primer, a second primer, and, optionally, a reverse transcriptase. In PCR techniques, the primers are designed to be complementary to sequences known to exist in a target nucleic acid to be amplified. Typically, the primers are chosen to be complementary to sequences that flank (and can be part of) the target nucleic acid sequence to be amplified. Preferably, the primers are chosen to be complementary to sequences that flank the target nucleic acid to be detected. Once the sequence of the target nucleic acid is known, the sequence of a primer is prepared by first determining the length or size of the target nucleic acid to be detected, determining appropriate flanking sequences that are near the 5′ and 3′ ends of the target nucleic acid sequence or close to the 5′ and 3′ ends, and determining the complementary nucleic acid sequence to the flanking areas of the target nucleic acid sequence using standard Watson-Crick base pairing rules, and then synthesizing the determined primer sequences. This preparation can be accomplished using any suitable methods known in the art, for example, cloning and restriction of appropriate sequences and direct chemical synthesis. Chemical synthesis methods can include, for example, the phosphotriester method described by Narang et al. (1979) Methods in Enzymology 68:90, the phosphodiester method disclosed by Brown et al. (1979) Methods in Enzymology 68:109, the diethylphosphoramidate method disclosed in Beaucage et al. (1981) Tetrahedron Letters 22:1859, and the solid support method disclosed in U.S. Pat. No. 4,458,066, all of which are incorporated herein by reference.

The ability of the first primer and second primer to form sufficiently stable hybrids to the target nucleic acid depends upon several factors, for example, the degree of complementarity exhibited between the primer and the target nucleic acid. Typically, an oligonucleotide having a higher degree of complementarity to its target will form a more stable hybrid with the target.

Additionally, the length of the primer can affect the temperature at which the primer will hybridize to the target nucleic acid. Generally, a longer primer will form a sufficiently stable hybrid to the target nucleic acid sequence at a higher temperature than will a shorter primer.

Further, the presence of high proportion of G or C or of particular non-natural bases in the primer can enhance the stability of a hybrid formed between the primer and the target nucleic acid. This increased stability can be due to, for example, the presence of three hydrogen bonds in a G-C interaction or other non-natural base pair interaction compared to two hydrogen bonds in an A-T interaction.

Stability of a nucleic acid duplex can be estimated or represented by the melting temperature, or “Tₘ.” The Tₘ of a particular nucleic acid duplex under specified conditions is the temperature at which 50% of the population of the nucleic acid duplexes dissociate into single-stranded nucleic acid molecules. The Tₘ of a particular nucleic acid duplex can be predicted by any suitable method. Suitable methods for determining the Tₘ of a particular nucleic acid duplex include oligo. Primers suitable for use in the methods and kits disclosed herein can be predetermined based on the predicted Tₘ of an oligonucleotide duplex that comprises the primer.

When the first primer and second primer are annealed to the target nucleic acid, a gap exists between the 3′ terminal nucleotide of the first primer and the 3′ terminal nucleotide of the second primer. The gap comprises a number of nucleotides of the target nucleic acid. The gap can be any number of nucleotides provided that the polymerase can effectively incorporate nucleotides into an elongating strand to fill the gap during a round of the PCR reaction (e.g., a round of annealing, extension, denaturation). Typically, a polymerase can place about 30 to about 100 bases per second. Thus, the maximum length of the gap between primers depends upon the amount of time within a round of PCR where the temperature is in a range in which the polymerase is active and the primers are annealed.

In accordance with the present invention, primers specifically hybridize to HCV nucleic acid. In some embodiments, the primers are specific to the 3′ NC region. Exemplary primers specific to the 3′ NC region of SEQ ID NO: 1 are indicated in Table 1.

TABLE 1
Exemplary Primer Sequences

| | | | |
|---|---|---|---|
| Name | SEQ ID NO | Sequence | Description |

| | | | |
|---|---|---|---|
| SCJ3721 | SEQ ID NO: 2 | agCCCTAGTCACGGCTAGC | HCV For 61.0/53.7/65.9 |
| SCJ3722 | SEQ ID NO: 3 | cacGTCACGGCTAGCTGTGA | HCV For 61.0/52.0/66.8 |
| SCJ3723 | SEQ ID NO: 4 | taggCTCCATCTTAGCCCTAGTC | HCV For 57.9/53.0/64.5 |
| SCJ3724 | SEQ ID NO: 5 | tcacTCACGGCTAGCTGTGAAAG | HCV For 61.9/52.0/66.5 |
| SCJ3725 | SEQ ID NO: 6 | gacctGCTAGCTGTGAAAGGTCC | HCV For 59.7/49.2/66.9 |
| SCJ3726 | SEQ ID NO: 7 | cgatATGGTGGCTCCATCTTAG | HCV For 58.1/55.1/62.2 |
| SCJ3727 | SEQ ID NO: 8 | acgGCTGTGAAAGGTCCGTG | HCV For 60.3/53.5/67.4 |
| SCJ3728 | SEQ ID NO: 9 | gctaGGCTCCATCTTAGCCC | HCV For 58.3/53.0/64.2 |
| SCJ3729 | SEQ ID NO: 10 | FAM-XcgGCAGTCATGCGGCT | HCV Rev 58.8/47.3/65.6 |
| SCJ3730 | SEQ ID NO: 11 | FAM-XaaagGCTCACGGACCTTTCA | HCV Rev 58.3/48.0/64.0 |
| SCJ3731 | SEQ ID NO: 12 | FAM-XgctgACGGACCTTTCACAGCTA | HCV Rev 60.2/48.2/66.5 |
| SCJ3732 | SEQ ID NO: 13 | FAM-XagCATGCGGCTCACGG | HCV Rev 59.8/51.1/65.6 |
| SCJ3733 | SEQ ID NO: 14 | FAM-XgaaagCTCACGGACCTTTCACAG | HCV Rev 59.5/43.3/65.6 |
| SCJ3734 | SEQ ID NO: 15 | FAM-XaggtCGGCTCACGGACCTT | HCV Rev 60.8/54.8/68.0 |

X = iso-C

### Non-Natural Bases

As contemplated in the methods and kits disclosed herein, at least one primer typically comprises at least one non-natural base. DNA and RNA are oligonucleotides that include deoxyriboses or riboses, respectively, coupled by phosphodiester bonds. Each deoxyribose or ribose includes a base coupled to a sugar. The bases incorporated in naturally-occurring DNA and RNA are adenosine (A), guanosine (G), thymidine (T), cytosine (C), and uridine (U). These five bases are “natural bases”. According to the rules of base pairing elaborated by Watson and Crick, the natural bases can hybridize to form purine-pyrimidine base pairs, where G pairs with C and A pairs with T or U. These pairing rules facilitate specific hybridization of an oligonucleotide with a complementary oligonucleotide.

The formation of these base pairs by the natural bases is facilitated by the generation of two or three hydrogen bonds between the two bases of each base pair. Each of the bases includes two or three hydrogen bond donor(s) and hydrogen bond acceptor(s). The hydrogen bonds of the base pair are each formed by the interaction of at least one hydrogen bond donor on one base with a hydrogen bond acceptor on the other base. Hydrogen bond donors include, for example, heteroatoms (e.g., oxygen or nitrogen) that have at least one attached hydrogen. Hydrogen bond acceptors include, for example, heteroatoms (e.g., oxygen or nitrogen) that have a lone pair of electrons.

The natural bases, A, G, C, T, and U, can be derivatized by substitution at non-hydrogen bonding sites to form modified natural bases. For example, a natural base can be derivatized for attachment to a support by coupling a reactive functional group (for example, thiol, hydrazine, alcohol, amine, and the like) to a non-hydrogen bonding atom of the base. Other possible substituents include, for example, biotin, digoxigenin, fluorescent groups, alkyl groups (e.g., methyl or ethyl), and the like.

Non-natural bases, which form hydrogen-bonding base pairs, can also be constructed as described, for example, in U.S. Pat. Nos. 5,432,272; 5,965,364; 6,001,983; 6,037,120; 6,140,169; and U.S. published application no. 2002-0150900, all of which are incorporated herein by reference. Suitable bases and their corresponding base pairs may include the following bases in base pair combinations (iso-C/iso-G, K/X, H/J, and M/N):

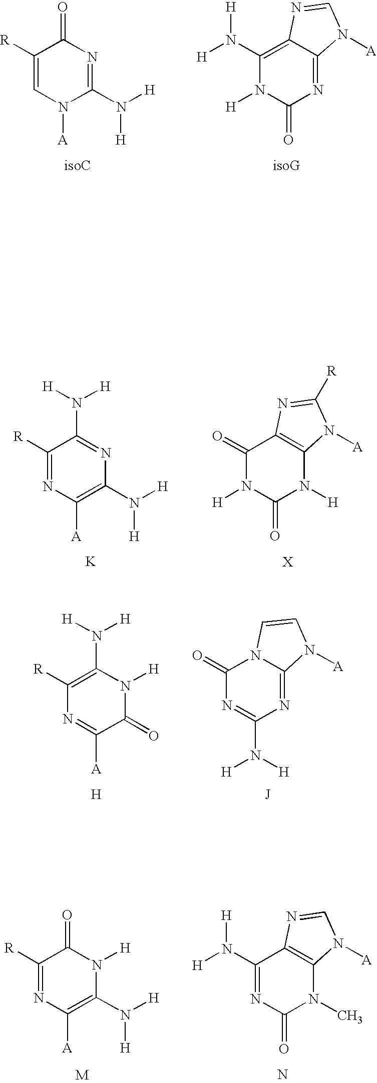

where A is the point of attachment to the sugar or other portion of the polymeric backbone and R is H or a substituted or unsubstituted alkyl group. It will be recognized that other non-natural bases utilizing hydrogen bonding can be prepared, as well as modifications of the above-identified non-natural bases by incorporation of functional groups at the non-hydrogen bonding atoms of the bases.

The hydrogen bonding of these non-natural base pairs is similar to those of the natural bases where two or three hydrogen bonds are formed between hydrogen bond acceptors and hydrogen bond donors of the pairing non-natural bases. One of the differences between the natural bases and these non-natural bases is the number and position of hydrogen bond acceptors and hydrogen bond donors. For example, cytosine can be considered a donor/acceptor/acceptor base with guanine being the complementary acceptor/donor/donor base. Iso-C is an acceptor/acceptor/donor base and iso-G is the complementary donor/donor/acceptor base, as illustrated in U.S. Pat. No. 6,037,120, incorporated herein by reference.

Other non-natural bases for use in oligonucleotides include, for example, naphthalene, phenanthrene, and pyrene derivatives as discussed, for example, in Ren et al., J. Am. Chem. Soc. 118, 1671 (1996) and McMinn et al., J. Am. Chem. Soc. 121, 11585 (1999), both of which are incorporated herein by reference. These bases do not utilize hydrogen bonding for stabilization, but instead rely on hydrophobic or van der Waals interactions to form base pairs.

As mentioned above, the polymerase can, in some instances, misincorporate a base opposite a non-natural base. In this embodiment, the misincorporation takes place because the reaction mix does not include a complementary non-natural base. Therefore, if given sufficient amount of time, the polymerase can, in some cases, misincorporate a base that is present in the reaction mixture opposite the non-natural base.

### Amplification

During PCR, the polymerase enzyme, first primer and second primer are used to generate an amplification product as described herein. Traditional PCR methods include the following steps: denaturation, or melting of double-stranded nucleic acids; annealing of primers; and extension of the primers using a polymerase. This cycle is repeated by denaturing the extended primers and starting again. The number of copies of the target sequence in principle grows exponentially. In practice, it typically doubles with each cycle until reaching a plateau at which more primer-template accumulates than the enzyme can extend during the cycle; then the increase in target nucleic acid becomes linear.

As used herein, when referring to “steps” of PCR, a step is a period of time during which the reaction is maintained at a desired temperature without substantial fluctuation of that temperature. For example, the extension step for a typical PCR is about 30 seconds to about 60 seconds. The time for annealing and melting steps for a typical PCR can range from 30 seconds to 60 seconds. Additionally, the limit of how quickly the temperature can be changed from the annealing temperature to the melting temperature depends upon the efficiency of the polymerase in incorporating bases onto an extending primer and the number of bases it must incorporate, which is determined by the gap between the primers and the length of the primers. The number of amplification cycles required to determine the presence of a nucleic acid sequence in a sample can vary depending on the number of target molecules in the sample. In one of the examples described below, a total of 50 cycles was adequate.

### Labels

In accordance with the methods and kits disclosed herein, the specific primers and the added non-natural nucleotide base may comprise a label. Nucleotides and oligonucleotides can be labeled by incorporating moieties detectable by spectroscopic, photochemical, biochemical, immunochemical, or chemical assays. The method of linking or conjugating the label to the nucleotide or oligonucleotide depends on the type of label(s) used and the position of the label on the nucleotide or oligonucleotide.

In some situations, it is desirable to use two interactive labels on a single oligonucleotide with due consideration given for maintaining an appropriate spacing of the labels on the oligonucleotide to permit the separation of the labels during oligonucleotide hydrolysis. It can be similarly desirable to use two interactive labels on different molecules, such as, for example, the reporter and the second primer. In this embodiment, the reporter and the primer are designed to contact each other when the reporter is incorporated during amplification. Again, consideration is given to maintaining an appropriate spacing of the labels between the molecules. One type of interactive label pair is a quencher-dye pair. Preferably, the quencher-dye pair is comprised of a fluorophore and a quencher. The ordinarily skilled artisan can select a suitable quencher moiety that will quench the emission of the particular fluorophore. In the exemplified embodiment, the Dabcyl quencher absorbs the emission of fluorescence from the fluorophore moiety. Fluorophore-quencher pairs have been described in Morrison, Detection of Energy Transfer and Fluorescence Quenching in Nonisotopic Probing, Blotting and Sequencing Academic Press, 1995, incorporated herein by reference.

Alternatively, the proximity of the two labels can be detected using fluorescence resonance energy transfer (FRET) or fluorescence polarization. FRET is a distance-dependent interaction between the electronic excited states of two dye molecules in which excitation is transferred from a donor molecule to an acceptor molecule without emission of a photon. Examples of donor/acceptor dye pairs for FRET are known in the art and may include fluorophores and quenchers described herein such as Fluorescein/Tetramethylrhodamine, IAEDANS™/Fluorescein (Molecular Probes, Eugene, Oreg.), EDANS™/Dabcyl, Fluorescein/Fluorescein (Molecular Probes, Eugene, Oreg.), BODIPY™ FL/BODIPY™ FL (Molecular Probes, Eugene, Oreg.), and Fluorescein/QSY7™.

The labels can be conjugated to the nucleotides, including non-natural bases, or oligonucleotides directly or indirectly by a variety of techniques. Depending upon the precise type of label used, the label can be located at the 5′ or 3′ end of the reporter, located internally in the reporter's nucleotide sequence, or attached to spacer arms extending from the reporter and having various sizes and compositions to facilitate signal interactions. Using commercially available phosphoramidite reagents, one can produce oligonucleotides containing functional groups (e.g., thiols or primary amines) at either terminus, for example by the coupling of a phosphoramidite dye to the 5′ hydroxyl of the 5′ base by the formation of a phosphate bond, or internally, via an appropriately protected phosphoramidite, and can label them using protocols described in, for example, PCR Protocols: A Guide to Methods and Applications, ed. by Innis et al., Academic Press, Inc., 1990, incorporated herein by reference.

Methods for incorporating oligonucleotide functionalizing reagents having one or more sulfhydryl, amino or hydroxyl moieties into the oligonucleotide reporter sequence, typically at the 5′ terminus, are described in U.S. Pat. No. 4,914,210, incorporated herein by reference. For example, 5′ phosphate group can be incorporated as a radioisotope by using polynucleotide kinase and [γ³²P]ATP to provide a reporter group. Biotin can be added to the 5′ end by reacting an aminothymidine residue, introduced during synthesis, with an N-hydroxysuccinimide ester of biotin.

Labels at the 3′ terminus, for example, can employ polynucleotide terminal transferase to add the desired moiety, such as for example, cordycepin, ³⁵S-dATP, and biotinylated dUTP.

Oligonucleotide derivatives are also available as labels. For example, etheno-dA and etheno-A are known fluorescent adenine nucleotides which can be incorporated into a reporter. Similarly, etheno-dC is another analog that can be used in reporter synthesis. The reporters containing such nucleotide derivatives can be hydrolyzed to release much more strongly fluorescent mononucleotides by the polymerase's 5′ to 3′ nuclease activity as nucleic acid polymerase extends a primer during PCR.

### Incorporation of Non-Natural Bases

In some embodiments, a region of the first and/or second primer comprises a non-natural base. A non-natural base that is complementary to the non-natural base present in the first and/or second primer is incorporated into the amplification product using a suitable enzyme. In this embodiment, the incorporation of the non-natural base is correlated with the presence of the target nucleic acid in the sample.

The disclosed methods and kits may employ a reporter; a nucleic acid polymerase, a first primer, and a second primer. The PCR reaction mixture may include the four naturally occurring deoxynucleotide triphosphates (i.e., dATP, dCTP, dGTP, and dTTP) as well as one or more non-natural nucleotide triphosphates (or an oligonucleotide containing a non-natural nucleotide triphosphate) as the reporter. In some embodiments, the one or more non-natural nucleotide triphosphates in the reaction mixture comprises a label, which may include a dye and/or a quencher.

The first primer may comprise a sequence complementary to a portion of a target nucleic acid and can hybridize to that portion of the target nucleic acid. The second primer may have a first region and a second region. The first region may comprises a sequence complementary to a portion of the target sequence. The second region of the second primer may comprise a sequence that is not complementary to the target nucleic acid and may comprise at least one non-natural base. It will be understood that the second region can include additional nucleotides. Preferably, the non-natural base is located at the junction between the first region and the second region of the second primer. In some embodiments, the non-natural base present in the second region of the second oligonucleotide primer is an iso-C or an iso-G.

In addition to the first primer and second primer, the sample is reacted or contacted with a polymerase, and a polymerase chain reaction is performed. If the target nucleic acid is present in the sample, the complementary portion of the first primer and the complementary portion of the second primer anneal to the corresponding regions of the target nucleic acid following standard base-pairing rules. When the primers are annealed to the target, the 3′ terminal nucleotide of the first primer is separated from the 3′ terminal nucleotide of the second primer by a sequence of nucleotides, or a “gap.” In a preferred embodiment, the first and second primers are designed such that gap of between about zero (0) to about five (5) bases on the template nucleic acid exists between the 3′ ends of the PCR primers when annealed to the template nucleic acid.

The polymerase is used to synthesize a single strand from the 3′-OH end of each primer, using polymerase chain reaction. The polymerase chain reaction is allowed to proceed for the desired number of cycles, to obtain an amplification product. The amplification product may include a double-stranded region and a single-stranded region, which comprises the at least one non-natural base of the second primer.

The reporter may comprise a label and at least one non-natural base. The reporter may be incorporated into the amplification product opposite the non-natural base. In some embodiments, the non-natural base of the reporter comprises a nucleotide triphosphate base that is complementary to the non-natural base of the single-stranded region of the amplification product. In this embodiment, the PCR reaction includes the presence of labeled non-natural nucleotide triphosphate base, in addition to the four naturally occurring nucleotide triphosphate bases (i.e., dATP, dCTP, dGTP, and dTTP). The concentration of non-natural nucleotide triphosphate base in the PCR reaction can range, for example, from 1 μM to 100 μM. The non-natural nucleotide triphosphate base may include a label.

Suitable enzymes for incorporation of the reporter into the amplification product include, for example, polymerases and ligases. A number of polymerases that are capable of incorporating natural nucleotides into an extending primer chain can also incorporate a non-natural base into an amplification product opposite a complementary non-natural base. Typically, class A DNA polymerases; such as Klenow, Tfl, Tth, Taq, Hot Tub, and Bst, are better able than class B polymerases; such as Pfu, Tli, Vent exo-, T4, and Pwo, to incorporate a non-natural base. Reverse transcriptases, such as HIV-1 reverse transcriptase, can also be used to incorporate non-natural bases into an extending primer opposite its complementary non-natural base within a template. In this embodiment the polymerase can be nuclease deficient or can have reduced nuclease activity. While not intended to limit the disclosed methods and kits, nuclease deficient polymerases are expected to be more robust because nuclease activities have been shown to interfere with some PCR reactions (Gene 1992 112(1):29-35 and Science 1993 260(5109):778-83).

Presence of the target nucleic acid in the sample is determined by correlating the presence of the reporter in the amplification product. Suitable detection and visualization methods are used to detect the target nucleic acid. For example, presence of the target nucleic acid may be determined by detecting the label by fluorescence or other visualization method. Fluorescence polarization, for example, can be used to detect the incorporation of the reporter into the amplification product.

In some embodiments, a washing step or a separation step is performed after incorporation of the reporter into the amplification product, and prior to detection. This washing or separation step will remove unbound or unincorporated reporter from the system, so that detection of signal is dependent upon incorporated reporter. One of skill in the art would readily appreciate that any known washing or separation steps can be used in connection with the disclosed methods and kits, including size separation by gel electrophoresis, and the like. Alternatively, a washing step may not be needed when fluorescence polarization is used as the method of detection.

The reporter used in this embodiment comprises at least one non-natural base. The non-natural base(s) of the reporter preferably include a label. The non-natural base(s) of the reporter is capable of being inserted by the polymerase into the amplification product opposite to the at least one non-natural base of the second primer during the PCR amplification.

In other embodiments, the reporter comprises a non-natural base (which is complementary to a non-natural base present in the first and/or second primer), and a quencher. In this embodiment, the non-natural base of the first and/or second primer includes a dye. In this embodiment, incorporation of the reporter brings the quencher into proximity with the dye. This, in turn, reduces the signal output of the dye, and this reduction in signal can be detected and correlated with the presence of the target nucleic acid. Suitable dye-quencher pairs are discussed above. Alternatively, a dye-dye pair can be used for fluorescence induction. When the target nucleic acid is present, PCR creates a duplexed product that places the two dyes in close proximity, and the fluorescent output of the label changes. The change is detectable by bench-top fluorescent plate readers or using real time monitoring.

### Detection

In one embodiment, when the target is present, an amplification product is created that places the first and second labels (e.g. dye/dye pair or dye/quencher pair) into close proximity. When the two labels are in close proximity, the fluorescent output of the reporter molecule label changes. The change is detectable by most bench-top fluorescent plate readers or using real time monitoring/detection. In this embodiment, the fluorescent output of the reporter molecule label changes, and this change is detectable. Other suitable detection methods are contemplated for used in the disclosed methods and kits.

In some embodiments, amplification is performed and detected on an instrument capable of reading fluorescence during thermal cycling, i.e. “real-time PCR” or “real-time monitoring.” Real time PCR allows for the detection and quantitation of a nucleic acid target. Various instruments capable of performing real time PCR are known in the art and include, for example, ABI Prism® 7900 (Applied Biosystems) and LightCycler® systems (Roche).

The fluorescent signal generated at each cycle of PCR is proportional to the amount of PCR product. A plot of fluorescence versus cycle number is used to describe the kinetics of amplification and a fluorescence threshold level is used to define a fractional cycle number related to initial template concentration. Specifically, the log of the initial template concentration is inversely proportional to the threshold cycle number (C_{T}), defined as the intersection of the fluorescence versus cycle number curve with the fluorescence threshold. Higher amounts of starting template results in PCR detection at a lower C_{T} value, whereas lower amounts require a greater number of PCR cycles to achieve an equivalent fluorescent threshold (C_{T}) and are detected at higher C_{T} values. The C_{T} is when the system begins to detect the increase or decrease in the signal associated with an exponential growth of PCR product during the log-linear phase. The slope of the log-linear phase is a reflection of the amplification efficiency and bona fide amplification is indicated by an inflection point in the slope, the point on the growth curve when the log-linear phase beings. The point also represents the greatest rate of change along the growth curve. Thus, the setting of this fluorescence threshold is defined as a level that represents a statistically significant increase over background fluorescent noise.

In some embodiments, when amplification is performed and detected on an instrument capable of reading fluorescence during thermal cycling, the intended PCR product from non-specific PCR products can be differentiated. Amplification products other than the intended products can be formed when there is a limited amount of template nucleic acid. This can be due to a primer dimer formation where a primer is incorporated into a primer dimer with itself or another primer. During primer dimer formation the 3′ ends of the two primers hybridize and are extended by the nucleic acid polymerase to the 5′ end of each primer involved. This creates a substrate that when formed is a perfect substrate for the primers involved to exponentially create more of this non-specific products in subsequent rounds of amplification. Therefore, the initial formation of the primer dimer does not need to be a favorable interaction since even if it is a very rare event the amplification process can allow the dimer product to overwhelm the reaction, particularly when template nucleic acid is limited or absent. Primer dimer products may give a detectable fluorescent change upon melting, but they are typically shorter in length than the intended product and therefore have a lower melting temperature. Since the labels are held in close proximity across the duplex an event that would separate the two strands would disrupt the interaction of the labels. Increasing the temperature of the reaction which contains the reaction products to above the Tₘ of the duplexed DNAs of the primer dimer and intended product may melt the DNA duplex of the product and disrupt the interaction of the labels giving a measurable change in fluorescence. By measuring the change in fluorescence while gradually increasing the temperature of the reaction subsequent to amplification and signal generation it may be possible to determine the Tₘ of the intended product as well as that of the nonspecific product.

### Multiplex Detection

The invention provides a method of detecting a plurality of target nucleic acids in a sample containing or suspected of containing the plurality of target nucleic acids. Methods for detecting multiple nucleic acids such as multiplex methods are increasingly important in medical diagnostics. Typical multiplex methods utilize PCR amplification, and in particular, real-time quantitative PCR. Methods for detecting nucleic acids that utilize probes and primers typically involve labeling each probe or primer with a unique label (e.g., a fluorescent dye). Multiplexing methods that utilize fluorescent dyes are often called “color multiplexing” methods. These methods require an instrument for detecting fluorescence from the multiple fluorophores, such as the ABI Prism® 7900 (Applied Biosystems) or LightCycler® system (Roche). In accordance with the present invention, an HCV specific primer comprising a non-natural base and a fluorescent label maybe included in an amplification mixture with specific primers to additional target nucleic acids. Alternatively, multiple HCV primers specific for different genotypes may be included in an amplification mixture. These additional primers will also have a non-natural base, but different fluorescent labels. In a suitable embodiment, a HCV specific primer comprises the dye FAM, while the specific primer to a second target nucleic acid comprises the dye HEX. Any of the other dyes disclosed herein may be substituted.

Methods for detecting multiple nucleic acid based on melting temperature (“Tm”) typically utilize small binders such as intercalators. These methods often are called “Tm multiplexing” methods. Melting temperature analysis may include determining the melting temperature of a complex formed by a probe and the amplified target nucleic acid, or determining the melting temperature of the amplified target nucleic acid itself (i.e., determining the Tm of the amplicon). Intercalators for Tₘ analysis typically exhibit a change in fluorescence based on whether the detected nucleic acid is double-stranded or single-stranded. Because intercalating agents interact with double-stranded nucleic acids non-specifically, multiple detected products must be distinguished by criteria such as resolvable melting temperatures.

Methods for detecting multiple nucleic acids are useful in the field of diagnostics. For example, methods for detecting multiple targets simultaneously may be useful for the detection of HCV and HIV. There is a high prevalence of HIV/HCV coinfection and because of critical clinical management issues for coinfected persons, detection of both pathogens using a multiplex method is desirable. HCV nucleic acid could also be detected simultaneously with other blood-borne pathogens (such as HBV, HTLV, HIV), or other pathogens or disease states, including virulent bacteria, or other viruses.

In one embodiment, HCV nucleic acid is detected simultaneously with an internal control nucleic acid, such as 18s rRNA, GAPDH, or β-actin.

### Use in Detection of DNA Polymorphisms

The disclosed methods and kits are useful for detecting sequence variations in nucleic acid sequences. As used herein, “sequence variation” refers to differences in nucleic acid sequence between two nucleic acids. For example, a nucleic acid from two or more HCV genotypes or subtypes can vary in sequence by the presence of single base substitutions or deletions or insertions of one or more nucleotides. One example of sequence variation is DNA polymorphisms. In some embodiments, detection of a single nucleotide polymorphism (SNP) using PCR is performed. Specific reporters or primers are used which contain a specific label. For example, a system for detecting multiple target nucleic acids might include specific reporters or primers with labels having different colors (e.g., two different fluorescent labels, three different fluorescent labels, or four different fluorescent labels). In a system for detecting two targets with two fluorescent labels, the presence of either color may indicate the presence of the respective target in the sample and the presence of the combination of the two colors may indicate that both targets are present in the sample.

In some embodiments, two or more primers are designed to detect a single nucleotide variation (e.g., substitution, deletion, or insertion) as follows. One of the primers used typically comprises a specific primer and may comprise a non-natural base. In one embodiment, both of these features are provided by a single primer. Alternatively, the specific primer is a separate primer from the primer that comprises a non-natural base.

The specific primers that can be used to discriminate the target nucleic acids may be designed to be complementary to each target such that the polymorphic base of interest is positioned at the 3′ end of the primer or near the 3′ end of the primer (e.g., within 1, 2, 3, 4, or 5 bases of the 3′ end of the primer). High levels of target discrimination may be achieved in part by the limited ability of selected polymerases to extend a primer which has a nucleotide mismatch at its 3′ end or near its 3′ end (e.g., within 1, 2, 3, 4, or 5 bases of the 3′ end of the primer) relative to a non-specific target. Additionally, target discrimination can be accomplished by placing the mismatch at other positions in the specific primer. Generally, the specific position can be anywhere within the primer provided that the primer cannot specifically hybridize to the target or the polymerase cannot efficiently extend the primer if there is a mismatch. In suitable embodiments, the primers are chosen so that the mismatch sufficiently destabilizes hybridization of the specific primer to a non-specific target nucleic acid for the selected PCR conditions. In one embodiment, the specific position is within about 5 bases from the 3′-end of the primer. For example, the specific position can be at the 3′-terminal base of the primer. These alternate positions for the specific position in the primer can be used to achieve selective amplification in two primary ways: 1) by lowering the Tₘ of the primer so that it is not hybridized on the template DNA during thermal cycling for the polymerase to extend, or 2) by creating an unfavorable primer/template structure that the polymerase will not extend. Enhanced specificity may be achieved by selecting suitable amplification conditions. In suitable embodiments, the primers are designed to have the shortest sequence possible and a Tₘ of approximately 55-60° C.

### Kits

Reagents employed in the disclosed methods can be packaged into diagnostic kits or individual components such as analyte-specific reagents. Diagnostic kits include at least a first and second primer. In some embodiments the kit includes non-natural bases capable of being incorporated into an elongating oligonucleotide by a polymerase. In one embodiment, the non-natural bases are labeled. If the oligonucleotide and non-natural base are unlabeled, the specific labeling reagents can also be included in the kit. The kit can also contain other suitably packaged reagents and materials needed for amplification, for example, buffers, dNTPs, or polymerizing enzymes, and for detection analysis, for example, enzymes and solid phase extractants.

Reagents useful for the disclosed methods can be stored in solution or can be lyophilized. When lyophilized, some or all of the reagents can be readily stored in microtiter plate wells for easy use after reconstitution. It is contemplated that any method for lyophilizing reagents known in the art would be suitable for preparing dried down reagents useful for the disclosed methods.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

### EXAMPLES

The present methods and kits, thus generally described, will be understood more readily by reference to the following examples, which are provided by way of illustration and are not intended to be limiting of the present methods and kits.

### Introduction

In one embodiment, a quantitative real-time PCR assay targets the 3′ NC domain of the HCV genome. The assay is based on the MultiCode®-RTx technology (EraGen Biosciences, Madison, Wis.). A Dabcyl quencher is covalently attached to iso-G triphosphate, which is site-specifically incorporated opposite an iso-C base, adjacent to a 5′ fluorescent label in one of the primers. MultiCode®-RTx does not require probes because specific amplification during PCR can be measured by a decrease in fluorescence. The lack of probes promotes higher sensitivity and a broader linear dynamic range for virtually any qualitative or quantitative assay.

The Bayer Versant® HCV RNA (bDNA) 3.0 and Roche COBAS Amplicor HCV Monitor™ 2.0 assays have an established track record in this area, but the technology employed by each is limited. Theoretically, a real-time PCR approach should provide increased sensitivity and a greater dynamic range. Both the Bayer and Roche products target the 5′ non-coding (5′ NC) domain of the HCV genome. However, the methods for the present invention are a probe-free technology that is particularly well-suited for a target such as the HCV 3′ NC domain. Additionally, the post-amplification melt profile capability of the present methods provides a means to confirm positive amplification products, thus reducing false positive calls.

In the present examples, the assay was evaluated for sensitivity, dynamic range, genotype independence, and accuracy using the HCV RNA Linearity Panel and Worldwide HCV Performance Panel (BBI Diagnostics, West Bridgewater, Mass.). In addition, over 150 clinical specimens were analyzed and compared with those generated from two commercial HCV quantitative methods. The results demonstrate that methods of detecting and quantifying HCV viral load described herein meet requisite performance criteria to be used to accurately measure HCV viral load.

### Methods

In vitro Transcript. A synthetic DNA molecule containing a T7 RNA polymerase promoter and the HCV 5′ and 3′ NC domains separated by a spacer region was used to generate a positive strand in vitro transcript (IVT). The recovered transcript was quantitated at OD₂₆₀ and copy number was determined based on the molecular weight of the RNA molecule.

Specimen Acquisition. Primary serum samples were obtained from a regional HCV treatment facility in St. Louis, Mo. The sources of these samples included naive, sero-positive, sero-negative, and HCV infected individuals during varying stages of therapeutic treatment. Each specimen was aliquoted and distributed for testing.

Specimen Processing. Each sample was monitored with an extractable control, an intact, inactivated RNA viral particle. Briefly, 5 μL of extraction control was added to 135 μL serum. RNA from the 140 μL sample was recovered using the Qiagen Viral RNA Mini Kit according to the manufacturer's instructions, with a final elution volume of 67.5 μL.

Amplification. For each sample the following 25 μL reaction was run on the ABI Prism 7900: 5.0 μL EraGen ISOlution™ (5×) (10 mM BTP pH 9.1, 50 mM KCl, 2.4 mM MgCl₂, 0.3 mg/mL BSA, 0.01% Tween20, 0.1 mM dATP, 0.1 mM dCTP, 0.1 mM dGTP, 0.2 mM dUTP, 1 μM Dabcyl-diGTP, 4 μM Decoy); 1.0 μL HCV Primer Mix (25×); 0.5 μL Titanium Taq (50×); 0.5 μL MMLV-RT (25 U/μL); 13.0 μL Nuclease-Free Water; 5.0 μL Sample. HCV primers (600 nM each) included GGCTCCATCTTAGCCC (SEQ ID NO: 9) and FAM-(iC)GCTCACGGACCTTTCA (SEQ ID NO: 11). MHV viral RNA was added to the reaction as an internal control. Primers for MHV included HEX-(iC)GCTTCCAGTAATTTGGTCTTCT (SEQ ID NO: 16) and GCCTTACAACCAACTACTCC (SEQ ID NO: 17). Cycling was according to Table 2.

TABLE 2
PCR Cycling Parameters

| Step | Time | | Temperature | Repeat |
|---|---|---|---|---|
| Reverse Transcription | 5 | min | 50° C. | 1 |
| Hot Start | 2 | min | 95° C. | 1 |
| Denature | 5 | sec | 95° C. | 50 |
| Anneal | 10 | sec | 58° C. | |
| Extend | 20 | sec | 72° C. | |
| Melt | | | 60° C. to 95° C. | 1 |

Data Analysis. Raw data was exported from the ABI Prism 7900 and imported into MultiCode®-RTx Analysis Software (EraGen Biosciences). HCV positive samples were determined using the following criteria: real-time amplification curves crossing the amplification threshold set at 10 SD from the baseline and melt peaks within 80.3° C.±1° C. Quantitative values for HCV RNA copy number were determined using an external standard curve. This curve was generated by performing 2-fold serial dilutions of previously characterized, high titer HCV positive serum. The formula obtained from those data (HCV RNA copies/mL=−0.28*Ct+15.08) was used to generate a quantitative value for HCV viral load for each sample tested.

### Example 1

### Dynamic Range

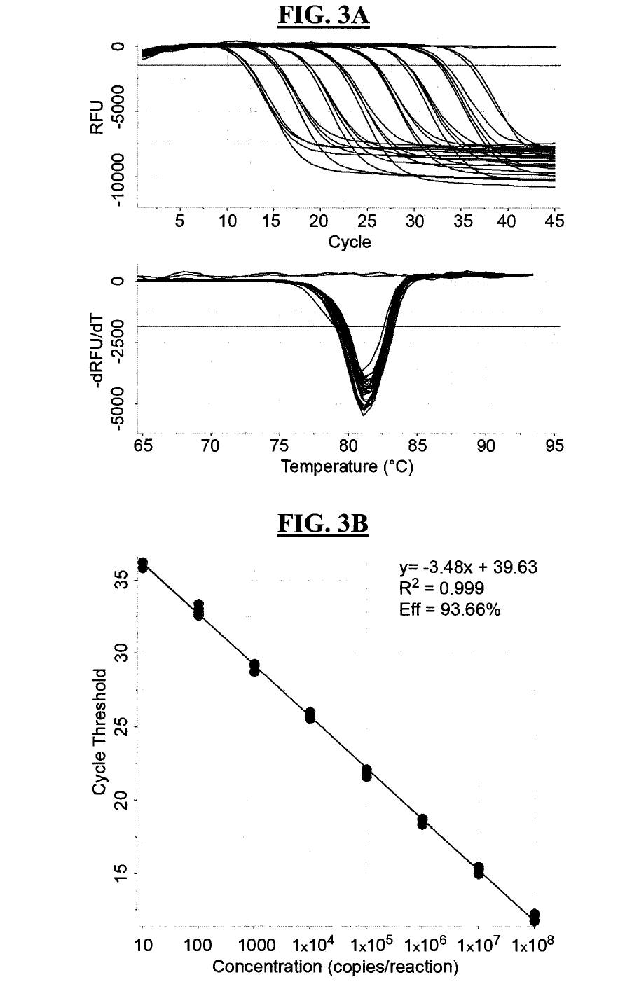

Dynamic range of the MultiCode-RTx HCV assay was tested using a 10-fold dilution series (HCV RNA copies/reaction) of IVT. The results are shown in FIGS. 3A and 3B. Four replicates of each concentration were tested (Red—10⁸, Orange—10⁷, Yellow—10⁶, Green—10⁵, Dark Blue—10⁴, Light Blue—10³, Purple—10², Pink—10¹). FIG. 3A (upper panel) shows that a statistically significant decrease in fluorescence occurs at an earlier cycle in those samples with higher concentration. A plot of Ct versus concentration (FIG. 3B) shows that amplification efficiency was 94% with an R² value of 0.999. At 10 HCV RNA copies/reaction, 2 out of 4 replicates were detected. Accordingly, the results indicate that HCV can be detected across 8 orders of magnitude. Melting curve analysis (FIG. 3A, lower panel) indicates that a specific amplification product was generated.

### Example 2

### Quantitative Analysis of 188 Specimens

Primary serum specimens from 188 patients were tested using three methods: MultiCode-RTx (as described in example 1 above), Bayer Versant® HCV RNA (bDNA) 3.0, and Roche COBAS Amplicor HCV Monitor™ 2.0. The Bayer and Roche methods produced 100% concordant results. Therefore, Bayer and Roche are presented as an aggregate.

The results comparing the detection assay with MultiCode®-RTx in accordance with the present invention to the Bayer/Roche assays are shown in Table 3. All 159 samples that were HCV positive by the Bayer and Roche methods were positive by MultiCode®-RTx, suggesting 100% sensitivity for MultiCode®-RTx.

TABLE 3
Assay Comparison

| | Bayer/Roche | Bayer/Roche |
|---|---|---|
| | Positive | Negative |
| MultiCode-RTx Positive | 159 | 10 |
| MultiCode-RTx Negative | 0 | 19 |

Table 3 above shows that 10 samples were HCV positive in the MultiCode®-RTx assay, but negative using the Bayer/Roche tests. These samples were then tested using the Roche COBAS TaqMan™ HCV test, which is another real-time PCR method. These results showing threshold cycle (Ct) for each discordant sample in the two different real-time PCR assays is indicated in Table 4. Eight of the ten samples were from individuals clinically diagnosed as HCV positive; one sample was seronegative for HCV and one sample was indeterminate. Six of ten of the putative “false positive” discordant samples also tested HCV positive using the Roche COBAS TaqMan™ test, suggesting that real-time PCR methods provide increased sensitivity over the other methods.

TABLE 4
Discordant Samples

| Sample | MultiCode- | COBAS | |
|---|---|---|---|
| ID | RTx Ct | TaqMan Ct | Clinical History |
| 63 | 39.8 | 39.4 | HCV |
| 115 | 39.5 | N/A | HCV |
| 129 | 42.7 | N/A | HCV |
| 130 | 39.9 | 39.5 | HCV |
| 131 | 41.7 | 40.3 | HCV |
| 170 | 37.4 | 41.4 | PSC¹ |
| 173 | 39.6 | N/A | PBC (Seronegative)² |
| 176 | 39.2 | N/A | HCV |
| 178 | 38.7 | 39.5 | HCV |
| 246 | 41.1 | 40.7 | HCV |

¹PSC: Primary sclerosing cholangitis (HCV status unknown)
²PBC: Primary biliary cirrhosis

### Example 3

### Quantitative Summary of HCV Positive Clinical Specimens

HCV RNA viral load was calculated for all the samples that tested positive in the examples above. The ability of the three methods (MultiCode®-RTx, Bayer Versant® HCV RNA bDNA 3.0, and Roche COBAS Amplicor HCV Monitor™ 2.0) to detect HCV over a range of concentrations was examined. Quantitative values are summarized by the number of samples detected within each log concentration range.

The results are shown in Table 5 and indicate that MultiCode®-RTx gives more positive calls and therefore greater sensitivity in the 10³-10⁵ copies/mL concentration range than either the Bayer or the Roche method. Values obtained by all three methods are similar in the 10⁵-10⁷ HCV RNA copies/mL range. MultiCode®-RTx and the Bayer method give more positive results in the 10⁷-10⁸ HCV RNA copies/mL concentration range.

TABLE 5
Assay Sensitivity at Various Concentrations

| HCV RNA | | | EraGen |
|---|---|---|---|
| (copies/mL) | Bayer Versant | Roche Amplicor | MultiCode-RTx |
| 10³-10⁴ | 0 | 0 | 6 |
| 10⁴-10⁵ | 3 | 2 | 8 |
| 10⁵-10⁶ | 24 | 22 | 25 |
| 10⁶-10⁷ | 90 | 117 | 76 |
| 10⁷-10⁸ | 42 | 18 | 54 |

### Example 4

### Deming Regression Analysis of HCV Viral Load

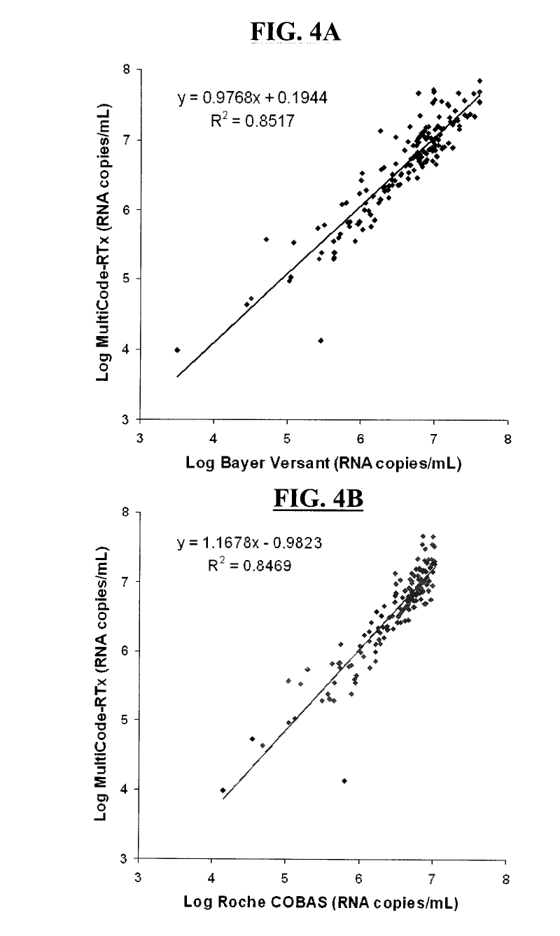

HCV viral load values for HCV positive clinical samples were obtained using: MultiCode®-RTx HCV system, Bayer Versant® HCV RNA bDNA 3.0, and Roche COBAS Amplicor HCV Monitor™ 2.0. Data were compared pairwise and fit to linear curves. A perfect match would give a slope of 1.0 and an intercept of zero. The results are shown in FIG. 4. FIG. 4A shows the MultiCode®-RTx HCV assay compared to Bayer Versant® HCV RNA bDNA 3.0. Samples below the dynamic range of the Bayer system (≦3,200 copies/mL) were excluded from the comparison. The data show a correlation (R²) of 0.8517. FIG. 4B shows the MultiCode-RTx HCV assay compared to Roche COBAS Amplicor HCV Monitor™ 2.0. Samples beyond the dynamic range of the Roche system (≦13,900 copies/mL or ≧11,500,000 copies/mL) were excluded from the comparison. The data show a correlation (R²) of 0.8469.

### Example 4

### Linearity and Sensitivity of MultiCode-RTx HCV Assay

An HCV RNA Linearity Panel (BBI Diagnostics, Bridgewater, Mass.) was obtained. Six samples were extracted with the extraction control as described previously. Each of these samples were tested using the MultiCode-RTx HCV assay (Green).

Quantitative values for the HCV RNA Linearity Panel were obtained using the Bayer Versant® HCV RNA bDNA 3.0 (Blue) and Roche COBAS Amplicor HCV Monitor™ 2.0 (Red) systems were provided by the manufacturer. Data were fit to a linear curve (FIG. 5).

### Example 5

### Genotype Independence of MultiCode®-RTx HCV Assay

Thirteen samples of six HCV genotypes in a Worldwide HCV Performance Panel (BBI Diagnostics, Bridgewater, Mass.) were obtained and extracted as described previously. HCV titers from the MultiCode®-RTx HCV System were compared to titers obtained using Bayer Versant® HCV RNA bDNA 3.0 and Roche COBAS Amplicor HCV Monitor™ 2.0.

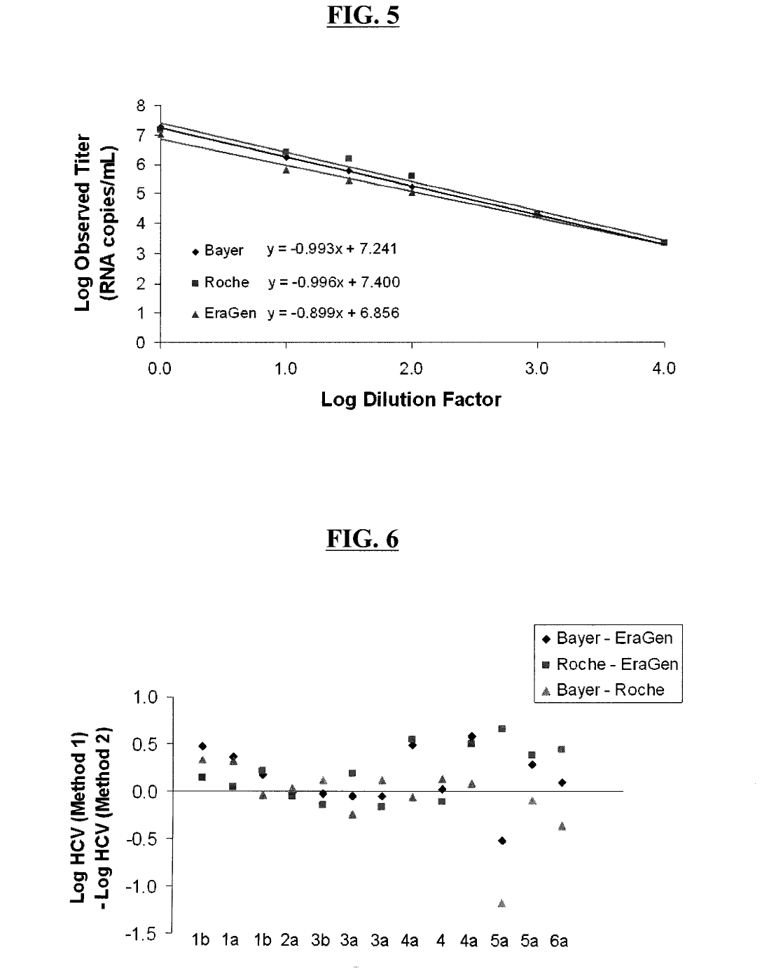

The results are shown in FIG. 6. HCV Titers were compared pairwise: Bayer-EraGen (Blue), Roche-EraGen (Red), Bayer-Roche (Green). The results indicate that all genotypes could be detected using the assay in accordance with the present invention. All samples, excepting one of genotype 5a, were quantified by MultiCode®-RTx within 0.6 logs of both comparator methods.

### Example 6

### Mixtures of Labeled and Unlabeled Primers to Enhance Assay Sensitivity

The detection assay according to the methods described above was performed using

HCV primers GGCTCCATCTTAGCCC (SEQ ID NO: 9) (600 nM) and FAM-(iC)GCTCACGGACCTTTCA (SEQ ID NO: 11). A comparison was made between a reaction comprising only labeled SEQ ID NO: 11 at 600 nM and a reaction comprising a 1:1 mixture of labeled and unlabeled SEQ ID NO: 11 (300 nM each). Samples were tested at various dilutions (5×LOD, 1×LOD, and 0.2×LOD). The results are shown in Tables 6 and 7 below and indicate that the mixture of labeled and unlabeled primers provided greater sensitivity at 0.2×LOD compared to reactions comprising only labeled primers.

TABLE 6
600 nM FAM Primer

| | Ave C_{T} | St Dev C_{T} | Ave Tₘ | St DevTₘ | % detection |
|---|---|---|---|---|---|
| 5X LOD | 35.20 | 0.60 | 81.18 | 0.11 | 100% |
| 1X LOD | 37.36 | 0.76 | 81.15 | 0.13 | 100% |
| 0.2X LOD | 39.95 | 2.62 | 81.17 | 0.10 | 68.75% |
| IAC | 27.15 | 0.30 | 75.54 | 0.12 | 100% |

TABLE 7
300 nM FAM Primer/300 nM unlabeled Primer

| | Ave C_{T} | St Dev C_{T} | Ave Tₘ | St DevTₘ | % detection |
|---|---|---|---|---|---|
| 5X LOD | 35.09 | 0.41 | 81.48 | 0.09 | 100% |
| 1X LOD | 37.54 | 0.80 | 81.37 | 0.07 | 100% |
| 0.2X LOD | 39.26 | 1.10 | 81.23 | 0.05 | 81.25% |
| IAC | 27.27 | 0.27 | 75.59 | 0.09 | 100% |

### CONCLUSIONS

This study shows that the 3′ NC domain of Hepatitis C Virus offers good clinical diagnostic utility, and can achieve results similar to or better than current HCV detection methods. Similar quantitative results were obtained for 188 clinical specimens using MultiCode®-RTx to target the 3′ NC as compared to Bayer Versant® HCV RNA bDNA 3.0 and Roche COBAS Amplicor HCV Monitor™ 2.0 assays that target the 5′ NC.

Ten additional HCV positive samples were detected at low levels and presumed below the limit of detection for the comparator methods. Six of these 10 samples were confirmed to be positive by the Roche COBAS TaqMan™ HCV test. Results for some samples that had saturated the high end of comparator methods had to be excluded from the Deming regression analysis. Therefore, the MultiCode®-RTx HCV assay offers a broader linear dynamic range with sensitivity exceeding that of the comparator methods.

Testing of proficiency panels with the MultiCode-RTx HCV assay demonstrated equivalent performance to that of Bayer and Roche assays. Furthermore, the MultiCode-RTx HCV assay exhibited genotype independence across all tested genotypes and subtypes, indicating the MultiCode-RTx HCV 3′ NC assay would be suitable for worldwide diagnostic applications.

1. A method comprising:
a) contacting a sample to be tested for the presence of HCV nucleic acid with
(i) at least one primer comprising a first label and a first non-natural base, wherein the at least one primer is capable of specifically hybridizing to a sequence from the 3′ NC region of the HCV nucleic acid; and
(ii) a reporter comprising a second label conjugated to a second non-natural base that base-pairs with the first non-natural base, wherein the first non-natural base is iso-C or iso-G and the second non-natural base is the other of iso-C or iso-G,
b) amplifying the HCV nucleic acid in the sample, if present, to generate amplification products, wherein the reporter is incorporated into the amplification products; and
c) observing a signal from the first label, the second label, or both to detect the HCV nucleic acid in the sample, if present.

2. The method of claim 1, wherein the at least one primer comprises a first primer and a second primer.

3. The method of claim 2, wherein the first primer comprises a sequence selected from the group consisting of SEQ ID NOS:2-9 and the second primer comprises a sequence selected from the group consisting of SEQ ID NOS:10-15 and 18.

4. The method of claim 2, wherein the first primer, the second primer, or both is capable of specifically hybridizing to a HCV nucleic acid from a HCV having a genotype selected from the group consisting of 1, 2, 3, 4, 5, and 6.

5. The method of claim 2, wherein the sample is contacted with a third primer, wherein the third primer has an sequence identical to the first primer and is unlabeled.

6. The method of claim 5, wherein the first and third primer are provided in equal amounts.

7. The method of claim 1, wherein the amplification products comprise a fragment of SEQ ID NO:1 or a nucleic acid having at least 95% sequence identity to a fragment of SEQ ID NO: 1.

8. The method of claim 1, wherein the first label comprises a fluorophore and the second label comprises a quencher.

9. The method of claim 8, wherein the fluorophore is selected from the group consisting of FAM, HEX, TAMRA, ROX, Cy3, Cy5, and Texas Red.

10. The method of claim 8, wherein the quencher is Dabcyl.

11. The method of claim 1, wherein observing the signal comprises observing a change in fluorescence of the fluorophore.

12. The method of claim 11, wherein the change in fluorescence is a decrease in fluorescence.

13. The method of claim 11, wherein the change in fluorescence is associated with a PCR cycle number.

14. The method of claim 13, further comprising the step of correlating the PCR cycle number to a starting copy number of HCV nucleic acid in the sample.

15. The method of claim 1, further comprising the step of determining a melting temperature of the amplification products.

16. A method comprising:
a) amplifying a HCV nucleic acid, if present in a sample, to provide an amplification product, wherein at least one primer used for amplifying the HCV nucleic acid comprises a first label and a first non-natural base, and at least one primer is capable of specifically hybridizing to the 3′ NC region of the HCV nucleic acid;
b) incorporating a reporter into the amplification product during amplification, wherein the reporter comprises a second label and a nucleotide triphosphate of a second non-natural base that base-pairs with the first non-natural base of the primer; wherein the first non-natural base is iso-C or iso-G and the second non-natural base is the other of iso-C or iso-G; and
c) observing a signal from at least one of the first label and second label during amplification thereby detecting and quantifying the HCV nucleic acid.

17. The method of claim 16, wherein at least one primer is capable of specifically hybridizing to a fragment of SEQ ID NO: 1.

18. The method of claim 16, wherein at least one primer is capable of specifically hybridizing to a HCV nucleic acid from a HCV having a genotype selected from the group consisting of 1, 2, 3, 4, 5, and 6.

19. The method of claim 16, wherein at least one primer comprises an oligonucleotide selected from any of SEQ ID NOS: 2-15 and 18.

20. The method of claim 16, further comprising the step of determining a melting temperature of the amplification products.


SEQUENCE LISTING (18 sequences)

SEQ ID NO: 1 - RNA, 99 nt, Hepatitis C virus
ugguggcuccaucuuagcccuagucacggcuagcugugaaagguccgugagcggcaugacugcagagagugcugauacuggccucucugcagaucaugu

SEQ ID NO: 2 - DNA, 19 bp, Artificial Sequence
  Description of Artificial Sequence Synthetic primer
agccctagtcacggctagc

SEQ ID NO: 3 - DNA, 20 bp, Artificial Sequence
  Description of Artificial Sequence Synthetic primer
cacgtcacggctagctgtga

SEQ ID NO: 4 - DNA, 23 bp, Artificial Sequence
  Description of Artificial Sequence Synthetic primer
taggctccatcttagccctagtc

SEQ ID NO: 5 - DNA, 23 bp, Artificial Sequence
  Description of Artificial Sequence Synthetic primer
tcactcacggctagctgtgaaag

SEQ ID NO: 6 - DNA, 23 bp, Artificial Sequence
  Description of Artificial Sequence Synthetic primer
gacctgctagctgtgaaaggtcc

SEQ ID NO: 7 - DNA, 22 bp, Artificial Sequence
  Description of Artificial Sequence Synthetic primer
cgatatggtggctccatcttag

SEQ ID NO: 8 - DNA, 20 bp, Artificial Sequence
  Description of Artificial Sequence Synthetic primer
acggctgtgaaaggtccgtg

SEQ ID NO: 9 - DNA, 20 bp, Artificial Sequence
  Description of Artificial Sequence Synthetic primer
gctaggctccatcttagccc

SEQ ID NO: 10 - DNA, 17 bp, Artificial Sequence
  Description of Artificial Sequence Synthetic primer
ncggcagtcatgcggct

SEQ ID NO: 11 - DNA, 21 bp, Artificial Sequence
  Description of Artificial Sequence Synthetic primer
naaaggctcacggacctttca

SEQ ID NO: 12 - DNA, 23 bp, Artificial Sequence
  Description of Artificial Sequence Synthetic primer
ngctgacggacctttcacagcta

SEQ ID NO: 13 - DNA, 17 bp, Artificial Sequence
  Description of Artificial Sequence Synthetic primer
nagcatgcggctcacgg

SEQ ID NO: 14 - DNA, 24 bp, Artificial Sequence
  Description of Artificial Sequence Synthetic primer
ngaaagctcacggacctttcacag

SEQ ID NO: 15 - DNA, 20 bp, Artificial Sequence
  Description of Artificial Sequence Synthetic primer
naggtcggctcacggacctt

SEQ ID NO: 16 - DNA, 23 bp, Artificial Sequence
  Description of Artificial Sequence Synthetic primer
ngcttccagtaatttggtcttct

SEQ ID NO: 17 - DNA, 20 bp, Artificial Sequence
  Description of Artificial Sequence Synthetic primer
gccttacaaccaactactcc

SEQ ID NO: 18 - DNA, 17 bp, Artificial Sequence
  Description of Artificial Sequence Synthetic primer
ngctcacggacctttca